(19) United States
(12) Patent Application Publication
(10) Pub. No.: US 2011/0086162 A1
(43) Pub. Date: Apr. 14, 2011

(54) Concentration Gradient Profiles For Control of Agent Release Rates From Polymer Matrices

(75) Inventors: Syed F.A. Hossainy, Fremont, CA (US); Fuh-Wei Tang, Temecula, CA (US); Andrew F. McNiven, Clonmel (IE); Joseph J. Eppert, Hemet, CA (US); Gregory J. Kevorkian, Temecula, CA (US)

(73) Assignee: ADVANCED CARDIOVASCULAR SYSTEMS, INC., Santa Clara, CA (US)

(21) Appl. No.: 12/955,828

(22) Filed: Nov. 29, 2010

Related U.S. Application Data
(63) Continuation of application No. 11/119,020, filed on Apr. 29, 2005.

(51) Int. Cl. A61L 33/06

(52) U.S. Cl. 427/2.25; 427/2.1; 427/2.24

(57) ABSTRACT

The present invention generally encompasses methods of coating which control of the release rate of agents from a polymeric matrix. This control over the release rate of agents provides for control over, inter alia, the therapeutic, prophylactic, diagnostic, and ameliorative effects that are realized by a patient in need of such treatment. In addition, the control of the release rate of agents also has an effect upon the mechanical integrity of the polymeric matrix, as well as a relationship to a subject's absorption rate of the absorbable polymers.

### CROSS-REFERENCE TO RELATED APPLICATIONS

This application is a continuation of co-pending U.S. patent application Ser. No. 11/119,020, filed on 29 Apr. 2005 and published as United States Patent Application Publication Number 2006-0246109 A1 on 2 Nov. 2006, which is incorporated by reference as if fully set forth, including any figures, herein.

### BACKGROUND

1. Field of the Invention

This invention is directed to the control of concentration gradients within polymeric matrices in the design of release profiles of agents from within these matrices.

2. Description of the State of the Art

Biomaterials research is continuously striving to improve the compositions from which medical devices and coatings are produced. For example, the control of protein adsorption on an implant surface and the local administration of agents from an implant are areas of focus in biomaterials research. Uncontrolled protein adsorption on an implant surface, for example, leads to a mixed layer of partially denatured proteins on the implant surface. This mixed layer of partially denatured proteins can lead to disease by providing cell-binding sites from adsorbed plasma proteins such as fibrinogen and immunoglobulin G. Platelets and inflammatory cells such as, for example, monocytes, macrophages and neutrophils, adhere to the cell-binding sites. A wide variety of proinflammatory and proliferative factors may be secreted and result in a diseased state. Accordingly, a non-fouling surface, which is a surface that does not become fouled or becomes less fouled with this layer of partially denatured proteins, is desirable.

A stent is an example of an implant that can benefit from improvements such as, for example, a non-fouling surface and a coating that can be used as a vehicle for delivering pharmaceutically active agents in a predictable manner. Stents can act as a mechanical intervention to physically hold open and, if desired, expand a passageway within a subject. Typically, a stent may be compressed, inserted into a small vessel through a catheter, and then expanded to a larger diameter once placed in a proper location. Examples of patents disclosing stents include U.S. Pat. Nos. 4,733,665, 4,800,882 and 4,886,062.

Stents play an important role in a variety of medical procedures such as, for example, percutaneous transluminal coronary angioplasty (PTCA), which is a procedure used to treat heart disease. In PTCA, a balloon catheter is inserted through a brachial or femoral artery, positioned across a coronary artery occlusion, inflated to compress atherosclerotic plaque and open the lumen of the coronary artery, deflated and withdrawn. Problems with PTCA include formation of intimal flaps or torn arterial linings, both of which can create another occlusion in the lumen of the coronary artery. Moreover, thrombosis and restenosis may occur several months after the procedure and create a need for additional angioplasty or a surgical by-pass operation. Stents are generally implanted to reduce occlusions, inhibit thrombosis and restenosis, and maintain patency within vascular lumens such as, for example, the lumen of a coronary artery.

Improvements to stents are also being developed to provide a controlled, local delivery of agents. Local delivery of agents is often preferred over systemic delivery of agents, particularly where high systemic doses are necessary to achieve an effect at a particular site within a subject—high systemic doses of agents can often create adverse effects within the subject. One proposed method of local delivery includes coating the surface of a medical article with a polymeric carrier and attaching an agent to, or blending it with, the polymeric carrier.

Agent-coated stents have demonstrated dramatic reductions in the rates of stent restenosis by inhibiting tissue growth associated with the restenosis. Restenosis is a very complicated process and agents have been applied in combination in an attempt to circumvent the process of restenosis. One method of applying multiple agents involves blending the agents together in one formulation and applying the blend to the surface of a stent in a polymer matrix. A disadvantage of this method is that the agents are released from the matrix through the blend and compete with one another for release.

The process of restenosis in coronary artery disease is derived from a complex interplay of several implant-centered biological parameters. These are thought to be the combination of elastic recoil, vascular remodeling, and neo-intimal hyperplasia. Since restenosis is a multifactorial phenomenon, the local agent delivery of agents from a stent would benefit from the design of a release rate profile that would deliver agents as needed from the stent in a controlled and predictable manner.

Unfortunately, the art has not yet developed a reliable way to control the release profile of agents from a medical device or coating, yet such control can be important to obtaining the desired effects or reducing any adverse effects that may otherwise occur from administration of the agents. In addition to providing a way to improve the bioactive, biobeneficial, and/or diagnostic results currently obtained from the administration of agents, control over the release rate of agents can assist in designing and maintaining the physical and mechanical properties of medical devices and coatings as well. Accordingly, control over the release of agents is an important design consideration and one of the next hallmarks in the development of stent technology.

### SUMMARY

The embodiments of the present invention generally encompass a medical device or coating comprising an agent, wherein the agent is distributed throughout a polymeric matrix in a predetermined initial concentration gradient profile (IC profile), wherein the IC profile was designed to provide a diffusion-controlled release of the agent from the polymeric matrix. In some embodiments, the medical device comprises a stent and the coating is on a stent.

In other embodiments, a method of creating a predetermined initial concentration gradient profile (IC profile) of an agent in a polymeric matrix is disclosed, wherein the method comprises selecting a release rate for an agent; preparing a composition comprising a polymer and the agent, wherein the composition was designed to provide a polymeric matrix with a desired diffusion coefficient for the agent; and forming the polymeric matrix from the composition, wherein the polymeric matrix comprises a predetermined IC profile of the agent, wherein the IC profile was designed to deliver the agent at the selected release rate from the polymeric matrix

### BRIEF DESCRIPTION OF THE FIGURES

FIG. 1 is a diagram used to illustrate the local pharmacokinetics of agent release from a stent and its subsequent uptake in the coronary vasculature according to some embodiments of the present invention.

FIG. 2 illustrates a cross-section of a coating on a stent strut within a vascular organ according to some embodiments of the present invention.

FIGS. 3a-3d illustrates initial concentration gradient profiles in a polymeric matrix according to some embodiments of the present invention.

FIG. 4 illustrates a coating comprising two distinct polymeric matrices containing a combination of agents with a combination of initial concentration gradient profiles according to some embodiments of the present invention.

FIG. 5 depicts an example of a three-dimensional view of a stent according to some embodiments of the present invention.

FIG. 6 illustrates select areas of an abluminal portion of a stent that can be selectively coated with a combination of agents using the IC profile designs according to some embodiments of the present invention.

FIGS. 7a and 7b illustrate a sandwiched-coating design according to some embodiments of the present invention.

FIG. 8 illustrates a checkerboard-type coating design by showing a top view of an abluminal surface of a stent that was coated in sections according to some embodiments of the present invention.

FIGS. 9a and 9b illustrate an engraved-type coating design by showing a top view of the abluminal surface of a stent with engravings according to some embodiments of the present invention.

FIG. 10 illustrates a section of a polymeric matrix containing an agent-enriched phase at a concentration that is below about 30% by volume according to some embodiments of the present invention.

FIG. 11 illustrates a section of a polymeric matrix containing an agent-enriched phase at a concentration that is above about 30% by volume according to some embodiments of the present invention.

FIGS. 12a and 12b illustrate an ejector assembly that does not require a nozzle, according to some embodiments of the present invention.

FIG. 13 demonstrates the accuracy of fit for an analytical model used to predict release rates of agents from polymeric matrices according to some embodiments of the present invention.

FIG. 14 shows the fraction of agent released as a function of time for three different coating configurations according to some embodiments of the present invention.

FIG. 15 shows the effect of agent-to-polymer ratios on agent release from a polymeric matrix according to some embodiments of the present invention.

FIG. 16 illustrates a graphical representation of a coating profile measurement that correlates point component concentration with depth according to some embodiments of the present invention.

FIGS. 17a and 17b illustrate a pictorial representation of a coating profile measurement that correlates bulk component concentration with position on the stent according to some embodiments of the present invention.

FIGS. 18a through 18d illustrate a pictorial representation of a coating profile measurement that correlates component distribution with depth according to some embodiments of the present invention.

### DETAILED DESCRIPTION

As discussed in more detail below, the embodiments of the present invention general encompass the control of the release rate of agents from a polymeric matrix. This control over the release rate of agents provides for control over, inter alia, the therapeutic, prophylactic, diagnostic, and ameliorative effects that are realized by a patient in need of such treatment. In addition, the control of the release rate of agents also has an effect upon the mechanical integrity of the polymeric matrix, as well as a relationship to a subject's absorption rate of the absorbable polymers.

An “agent” can be a moiety that may be bioactive, biobeneficial, diagnostic, plasticizing, or have a combination of these characteristics. A “moiety” can be a functional group composed of at least 1 atom, a bonded residue in a macromolecule, an individual unit in a copolymer or an entire polymeric block. It is to be appreciated that any medical devices that can be improved through the teachings described herein are within the scope of the present invention.

The compositions and methods of the present invention apply to the formation of medical devices and coatings. Examples of medical devices include, but are not limited to, stents, stent-grafts, vascular grafts, artificial heart valves, foramen ovale closure devices, cerebrospinal fluid shunts, pacemaker electrodes, guidewires, ventricular assist devices, cardiopulmonary bypass circuits, blood oxygenators, coronary shunts (AXIUS™, Guidant Corp.), vena cava filters, and endocardial leads (FINELINE® and ENDOTAK®, Guidant Corp.). In some embodiments, the stents include, but are not limited to, tubular stents, self-expanding stents, coil stents, ring stents, multi-design stents, and the like. In other embodiments, the stents are metallic; low-ferromagnetic; non-ferromagnetic; biostable polymeric; biodegradable polymeric or biodegradable metallic. In some embodiments, the stents include, but are not limited to, vascular stents, renal stents, biliary stents, pulmonary stents and gastrointestinal stents.

The medical devices can be comprised of a metal or an alloy, including, but not limited to, ELASTINITE® (Guidant Corp.), NITINOL® (Nitinol Devices and Components), stainless steel, tantalum, tantalum-based alloys, nickel-titanium alloy, platinum, platinum-based alloys such as, for example, platinum-iridium alloys, iridium, gold, magnesium, titanium, titanium-based alloys, zirconium-based alloys, alloys comprising cobalt and chromium (ELGILOY®, Elgiloy Specialty Metals, Inc.; MP35N and MP20N, SPS Technologies) or combinations thereof. The tradenames “MP35N” and “MP20N” describe alloys of cobalt, nickel, chromium and molybdenum. The MP35N consists of 35% cobalt, 35% nickel, 20% chromium, and 10% molybdenum. The MP20N consists of 50% cobalt, 20% nickel, 20% chromium, and 10% molybdenum. Medical devices with structural components that are comprised of bioabsorbable polymers or biostable polymers are also included within the scope of the present invention.

Generally speaking, there are numerous considerations in designing agent-release profiles for a polymer matrix including, but not limited to, the selection and characteristics of polymers and polymer combinations that form the polymeric matrix; the functional groups that are present on polymers in the matrix, either naturally or through modification; the selection of agents to combine with the polymers in a matrix; the polymorphism of the agents; the morphology of the polymeric matrix; the hydrophilicity/hydrophobicity of the polymeric matrix; and other process considerations selected for each step in the process, such as the temperature, pressure, humidity, solvent selection, etc., that exist in forming the compositions, forming the medical devices or coatings from the compositions, drying conditions, annealing conditions, and the like. The manner in which the agents are combined with the polymers can also have a profound effect such as, for example, whether the agents are bonded, blended, or a combination thereof, with the polymers. Interactions between the agents, polymers, and solvents can also affect the release profile of the agents.

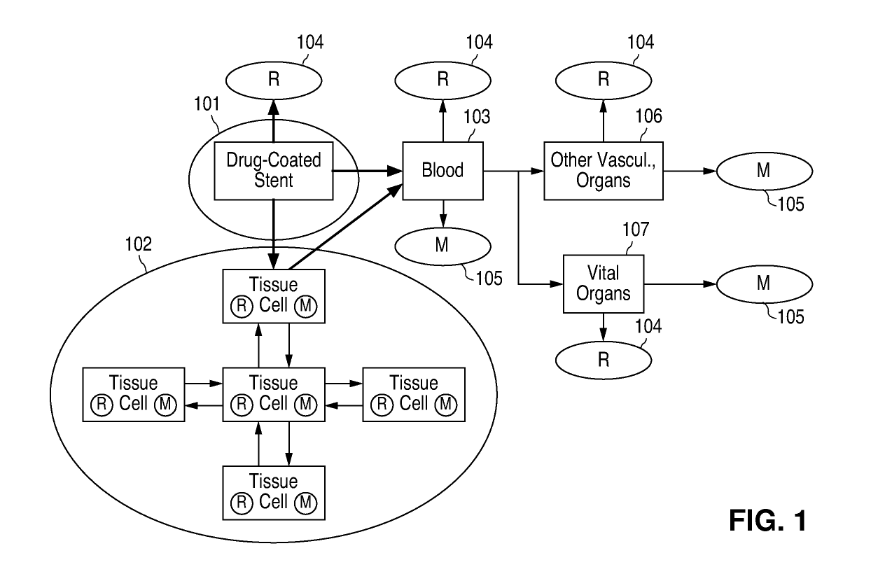

FIG. 1 is a diagram used to illustrate the local pharmacokinetics of agent release from a stent and its subsequent uptake in the coronary vasculature according to some embodiments of the present invention. In region 101, the agent that will be released from the stent is a drug. The agent can be released and passed through tissue cells within adjoining tissue 102, blood 103, or the agent can remain as residual agent (“R”) 104 on the stent. The agent can also be metabolized (“M”) 105 after its delivery to adjoining tissue 102, blood 103, other vascular organs 106, or vital organs 107.

FIG. 2 illustrates a cross-section of a coating on a stent strut within a vascular organ according to some embodiments of the present invention. The cross-section of the coated stent strut 201 includes a stent 202, an optional primer layer 203, a polymer reservoir 204 that includes at least one agent 205, and an optional top-coat layer 206 that can further control the diffusion of the agent 205 out of the polymer reservoir 204. The coated stent strut 201 is adjoining vascular tissue 207 and blood 208. The agent 205 is released from the polymer reservoir 204 into the blood 208 and the vascular tissue 207. This release of the agent 205 includes a diffusion parameter, so design of a polymeric matrix can include diffusion considerations in order to further obtain control over the release of the agent 205.

Diffusion Coefficients

The IC profile of an agent within a polymeric matrix provides a diffusion-controlled release of the agent within a subject. The process of diffusion of an agent from a stent can include, but is not limited to, the following four factors: (1) coating parameters, (2) coating process, (3) polymer physicochemical properties, and (4) agent physicochemical properties. The coating parameters include, but are not limited to, the initial solid phase concentration distribution, which includes the drug to polymer (D/P) ratio, the thickness of an agent-free polymer top-coating, the total drug content, the dispersed phase microstructure, and the like. The coating process includes, but is not limited to, the selection of solvents, the thermal history of processing, the thermodynamics of phase separation, the solution thermodynamics, kinetics, and the like. Polymer physicochemical properties include, but are not limited to, glass transition temperature (Tg), melting temperature (Tm), heat of fusion (ΔH_{f}), percent crystallinity, water absorption, lipid-induced swelling, and the like. Agent physicochemical properties include, but are not limited to, the degree and type of dispersed phase parameters, the extent of solid solution, the polymorphism of the agent (e.g. different crystalline forms of a drug), and the like.

Diffusion will occur wherever there is a diffusion medium such as, for example, the water that is taken up by a coating layer on a stent while implanted in a vascular organ. A mathematical expression is provided below to describe diffusion of an agent across a coating layer, where the driving force is the concentration gradient of the agent across the diffusion medium. The flux of the agent across the diffusion medium can be represented by the following formula:

$$F = {}^{-} D \frac{d C}{d x} , where \\ D = diffusion coefficient ( \frac{{L}^{2}}{t} ) ; \\ F = agent flux ( \frac{moles}{{L}^{2} * t} ) ; \\ \frac{d C}{d x} = Concentration gradient , i . e . , change in concentration \text{/} change in distance across the layer ( \frac{moles}{{L}^{4}} ) ; \\ L = any unit of layer dimension used , e . g . , to calculate area or thickness ; and \\ t = time . \qquad ( 1 )$$

As the agent travels through the coating layer, the flux of the agent changes with the concentration gradient. Starting from the general mass balance,

Input−Output+Generation=Accumulation, or

$${M}_{i} - {M}_{o} + {M}_{g} {=}^{-} D \frac{d Ci}{d x} {-}^{-} D \frac{d Co}{d x} \qquad ( 2 )$$

Using the mathematical relationship that

$${y}_{x} - {y}_{x} + d x = \frac{d y}{d x} d x ,$$

and assuming a constant diffusivity across the polymeric matrix of the coating layer, the relationship becomes

$$\frac{d M}{d x} d x + {M}_{g} {=}^{-} D \frac{{d}^{2} C}{d {x}^{2}} d x . \qquad ( 3 )$$

Since there is no generation of agent in the coating layer, M_{g}=0. Therefore,

$$\frac{d M}{d x} {=}^{-} D \frac{{d}^{2} C}{d {x}^{2}} ;$$

and, since

$$accumulation = \frac{d C}{d t} ,$$

the equation becomes Fick's Second Law:

$$\frac{d C}{d t} {=}^{-} D \frac{{d}^{2} C}{d {x}^{2}} . \qquad ( 4 )$$

Fick's Second Law tells us that the change in the concentration of the agent over time is equal to the change in the local flux of the agent. This provides a means to assess the rate of release of agents within particular polymeric matrix systems, wherein each system can have a number of factors that affect this rate of release. These factors have been presented above, and the net result of the combined diffusion-related factors within a given system can be cumulatively expressed as a diffusion coefficient. The diffusion coefficient can also be described as “effective-diffusion coefficient” for describing a particular system.

Without intending to be bound by any theory or mechanism of action, the diffusive transport of an agent can be divided into at least two modes referred to as “biphasic modes:”

(1) in a first mode, the effective diffusivity corresponds to the transport of an agent dissolved in a polymeric matrix without phase separation; or, an agent that primarily transports out of a dispersed agent phase into a surrounding polymeric matrix and then diffuses out of the surrounding polymeric matrix; and,

(2) in a second mode, the effective diffusivity corresponds to the transport of an agent through a dispersed agent phase, for example, a dispersed agent phase within a polymeric matrix that has interconnected to create a closely connected network (i.e. a “percolated” phase, which is discussed in more detail below) by virtue of being densely distributed throughout the polymeric matrix; accordingly, the effective diffusivity can include an intrinsic diffusivity of the agent through a water medium in the polymeric matrix in addition to the tortuosity and porosity of a percolated-phase passage that has formed throughout the polymeric matrix.

In some embodiments, the overall mass transport can be considered dependent on one or a combination of the biphasic modes. Since the diffusion coefficient can be directly proportional to the rate of release, it can be measured experimentally for each polymeric matrix system by one skilled in the art and used as a defining characteristic for agent release from within that system.

Initial Concentration Gradient Profiles

The embodiments of the present invention are directed to novel articles of manufacture such as, for example, a medical device comprising stent, wherein the stent includes a polymeric matrix having a predetermined initial concentration gradient profile (“IC profile”) of agents within the matrix. It has been discovered that these IC profiles can be designed to produce a controllable release rate of agents from a polymeric matrix. The “initial concentration gradient” refers to the concentration gradient of one or more agents across a polymeric matrix in its initial state after the medical device or coating has been manufactured but before implantation. The IC profile can refer to a profile in any direction or combination of directions across a polymeric matrix. In some embodiments, the IC profile can be an agent concentration across the thickness of a polymeric matrix from the air/polymer interface to the polymer/metal interface. In other embodiments, the IC profile can be an agent concentration throughout a polymeric matrix in any direction. In other embodiments, the IC profile can refer to the bulk concentration profile of an agent throughout a polymeric matrix in all directions.

Fick's Second Law tells us that the change in the concentration of the agent over time is equal to the change in the local flux of the agent. The derivation of Fick's Second Law provides some reasoning for an assumption that the diffusion-based flux of agents from a medical device or coating, i.e. diffusion-based release rate, may be controlled through the design of initial concentration gradients across the polymeric matrix used in the formation of the medical device or coating. Using such an assumption, a method of designing polymeric matrices having predetermined IC profiles of agents has been investigated as a way to predictably deliver agents in vivo from compositions used to form medical devices or coatings. The IC profiles can be mathematically described by some function, C=f(x), wherein the concentration of an agent at a particular point within a polymeric matrix depends on the location (x) of the agent across, for example, the thickness (L) of the polymeric matrix.

The IC profiles of the present invention can comprise a single function or any compilation of functions, where a “function” can be a mathematical representation, as described above, of at least a portion of an IC profile. In some embodiments, the function can comprise a linear portion such as, for example, a zero order function, a first order function, an exponential decay function, or a combination thereof. In other embodiments, the function can comprise a non-linear portion such as, for example, a second order function; a third order function; other polynomial function; an exponential function such as, for example, a growth function or a decay function; a logarithmic function such as, for example, a natural-logarithmic function (ln) or a base-10-logarithmic function (log₁₀); a power function; a wave function; a distribution function such as, for example, a normal distribution or a log-normal distribution, Poisson distribution, Weibull distribution, or a combination thereof. In other embodiments, the IC profile can comprise a linear portion and a non-linear portion.

The emphasis of the present invention is that virtually any IC profile or combination of IC profiles that represent a desired agent release can be designed across the polymeric matrix present in a medical device or a coating for a medical device. The use of a mathematical function provides a way to characterize a desired IC profile in the illustration and design of a process for creating desired IC profiles according to some embodiments of the present invention. The variety of initial concentration profiles that may be desired or may be designed is virtually limitless.

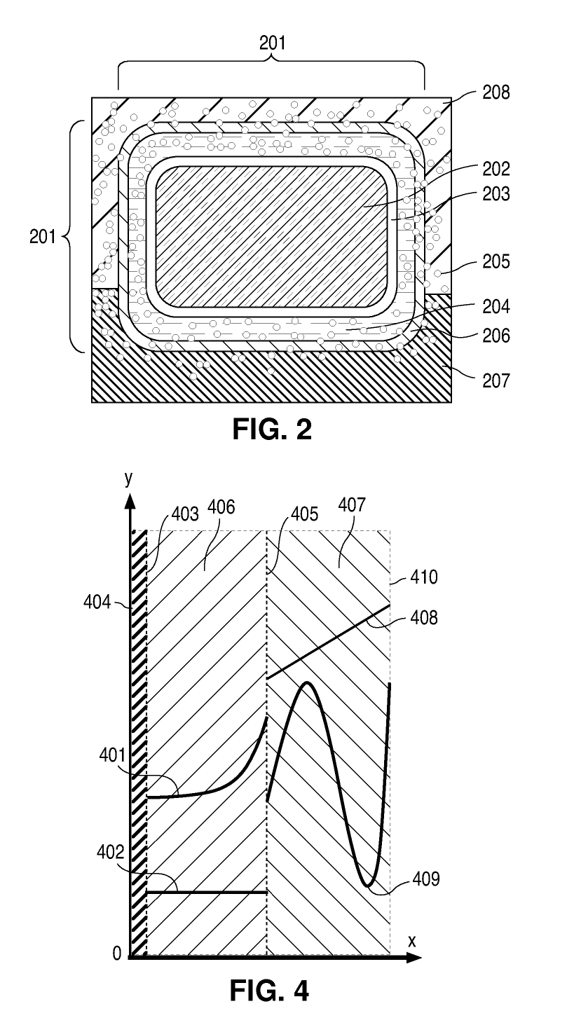

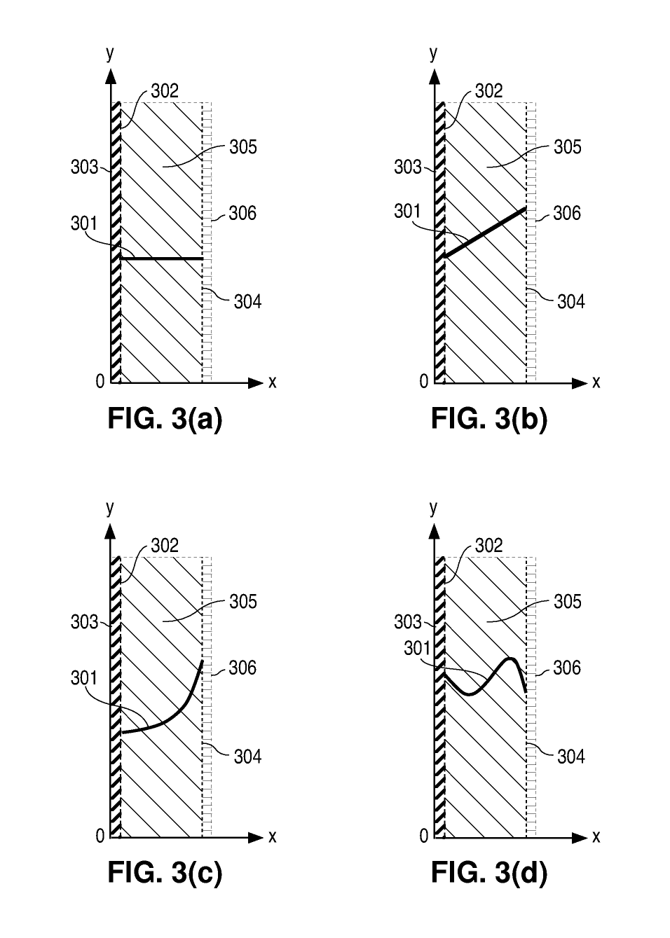

FIGS. 3a-3d illustrates initial concentration gradient profiles in a polymeric matrix according to some embodiments of the present invention. In FIGS. 3a-3d, the IC profile 301 begins at a boundary 302 at the surface 303 of a medical device and ends at a boundary 304 between the polymeric matrix 305 and an optional topcoat 306. In each of FIGS. 3a-3d, the profiles represent a correlation between the agent concentration on the y-axis and the position of the agent as measured from the boundary 302 of the surface 303 of the medical device on the x-axis. In FIG. 3a, the IC profile 301 is a linear profile, wherein the agent concentration is a zero order function of position in the polymeric matrix, and is a constant in this case. In FIG. 3b, the IC profile 301 is a linear profile, wherein the agent concentration is a first order function of position in the polymeric matrix. In FIG. 3c, the IC profile 301 is a non-linear profile, wherein the agent concentration is an exponential function of position in the polymeric matrix. In FIG. 3d, the IC profile 301 is a non-linear profile, wherein the agent concentration is a wave function of position in the polymeric matrix.

FIG. 4 illustrates a coating comprising two distinct polymeric matrices containing a combination of agents with a combination of initial concentration gradient profiles according to some embodiments of the present invention. The IC profiles 401 and 402 begin at a boundary 403 at the surface 404 of a medical device and end at a boundary 405 between a first polymeric matrix 406 and a second polymeric matrix 407. The IC profiles 408 and 409 begin at the boundary 405 between the first polymeric matrix 406 and the second polymeric matrix 407 and end at the outer surface 410 of the second polymeric matrix 407. In this embodiment, a combination of agents can be delivered, wherein each of the agents has its own diffusion coefficient for the polymeric matrix system through which the agent must pass. Accordingly, each of the agents follows its own IC profile to further control the rate of release of that agent from the polymeric matrices and provide a more exacting local delivery of agents within a subject. As with most embodiments of the present invention, an optional topcoat can be applied for further control of agent release, biocompatibility, or any other benefit or combination of benefits known to one of skill in the art that can be obtained using a topcoat.

In some embodiments, a polymeric matrix having one or more IC profiles can be applied as a uniform layer on the surface of a medical device or coating. In other embodiments, one or more polymeric matrices having one or more IC profiles can be applied to select regions on the surface of a medical device or coating. In other embodiments, a combination of polymeric matrices containing one or more IC profiles can be applied in predetermined patterns on the surface of a medical device or coating.

In many embodiments, the coating can be include depots or patterns as described in U.S. Pat. No. 6,395,326, which is incorporated herein by reference. In some embodiments, predetermined geometrical patterns can be deposited by moving a dispenser assembly, such as an acoustic ejector assembly, along a predetermined path while depositing the composition onto a stationary medical device such as, for example, a prosthesis or a stent. In other embodiments, the predetermined geometrical pattern can be deposited using a method that includes moving an assembly supporting the device along a predetermined path, while a stationary dispenser assembly deposits one or more compositions onto the device. In other embodiments, both the assembly supporting the device and the dispenser assembly can move to form the predetermined pattern on the device.

The predetermined geometrical pattern of the coating composition may be applied as a continuous stream that is either in a substantially straight line or a line that has a curved or angular pattern. The predetermined geometrical pattern may also be an intermittent pattern that is in a straight line, a line that curved or angular, and includes at least one agent or a combination of agents.

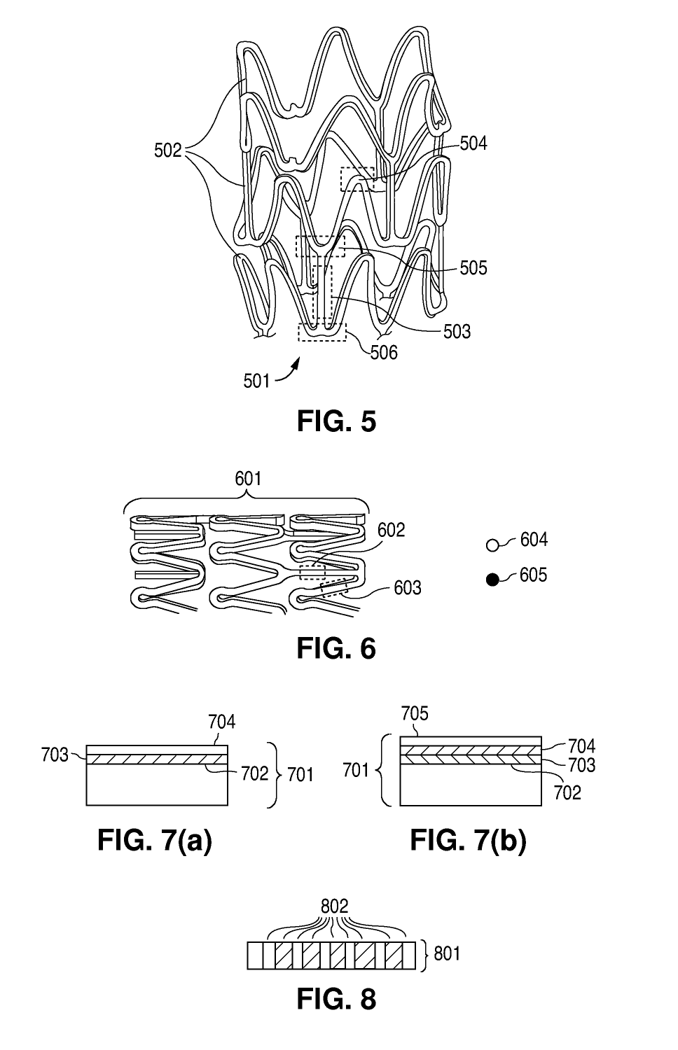

Embodiments of the devices described herein may be illustrated by a stent. FIG. 5 depicts an example of a three-dimensional view of a stent according to some embodiments of the present invention. The stent 501 may be made up of a pattern of a number of interconnecting structural elements or struts 502. As described herein, the embodiments disclosed are not limited to stents or to the stent pattern illustrated in FIG. 5 and are easily applicable to other patterns and other devices. The variations in the structure of patterns are virtually unlimited.

Designing predetermined IC profiles of the agents within the polymeric matrices can assist in obtaining and maintaining desirable physical and mechanical properties and, thus, aid in preventing failure within medical devices or coatings. Since many medical implants undergo a great deal of strain during their manufacture and use that can result in structural failure, the ability to apply particular polymeric matrices having particular agents to select regions can be invaluable to the success of a medical procedure. Structural failure can occur, for example, as a result of manipulating an implant in preparation for placing the implant in a subject and while placing the implant in a desired location in a subject. A stent is an example of an implant that may be compressed, inserted into a small vessel through a catheter, and then expanded to a larger diameter in a subject. Controlled application of particular agents in low strain areas 503 and high strain areas 504, 505, and 506 of a stent, for example, can help to avoid problems, such as cracking and flaking, that can occur during implantation of the stent.

In other embodiments, the agent-containing compositions can be applied selectively to an abluminal surface of a medical device such as, for example, a stent. In most embodiments, the stent can be an balloon-expandable stent or a self-expandable stent. The “abluminal” surface refers to the surface of the device that is directed away from the lumen of the organ in which the device has been deployed. In one example the lumen is an arterial lumen, and the abluminal surface of the stent is the surface that is placed in contact with the inner wall of the artery. Designing and applying predetermined IC profiles of agents within polymeric matrices to the abluminal surface of a medical device can provide a way for one of skill in the art to control the delivery of the agents within a subject and, thus, aid in preventing adverse effects and promoting desirable effects obtained from the agents.

FIG. 6 illustrates select areas of an abluminal portion of a stent that can be selectively coated with a combination of agents using the IC profile designs according to some embodiments of the present invention. In this embodiment, an IC profile for agent A 604 can be selectively applied to area 602, and an IC profile for agent B 605 can be selectively applied to area 603. This selective application of agents allows for a controlled release of each agent by allowing for the independent selection of the manner in which each agent is attached to a surface of the stent 601. For example, an agent may be combined with a polymer matrix as a blend, a chemical conjugation, or a combination thereof, which affects the rate of release. The agent may also be sandwiched between polymer layers, encapsulated within a polymer network, or any combination thereof, thereby providing a desired agent concentration such as, for example, a spike in agent concentration at the boundary of a polymeric matrix.

The embodiments for the IC profiles that are taught herein are not meant to be limiting. Other functions and combinations of functions for the IC profiles are possible and are virtually limitless in variety in the practice of the invention.

In some embodiments, a medical device can comprise a polymeric matrix having a predetermined release rate of one or more agents based on one or more select IC profiles. In other embodiments, a medical device can be coated with a composition comprising a polymeric matrix having a predetermined release rate of one or more agents based on one or more select IC profiles. In other embodiments, the medical device and coating can each have their own IC profiles, such that each profile is designed to release an agent at a predetermined rate.

In some embodiments, the polymeric matrix can release agents without biodegradation of the matrix, such that the agent-release design is at least partially independent of biodegradation. In other embodiments, the polymeric matrix releases agents during biodegradation of the matrix, such that the agent-release design is at least partially dependent on biodegradation. In other examples, the polymeric matrix releases agents according to a combination of IC profile designs, wherein the combination can include profiles that are at least partially independent of, or at least partially dependent on, biodegradation of the polymeric matrix.

In some embodiments, the medical device includes a stent, wherein the thickness of the struts that form the structure of the stent can be referred to as a layer or, in some embodiments, a combination of layers. In other embodiments, a combination of layers can be incrementally formed such as, for example, during the stacking of layers in a layered manufacturing process, the methods of which are known to those skilled in the art. In other embodiments, a layer or combination of layers can be applied as a coating on a surface of a medical device such as, for example, a stent. In other embodiments, the layers can be applied as a coating on select surfaces such as, for example, the abluminal surface of a stent. In other embodiments, the layers can be applied in predetermined geometrical patterns on select surfaces of a medical device such as, for example, a stent.

In other embodiments, each layer can be applied incrementally in controlled volumes such as, for example, through the use of an apparatus that ejects controlled volumes of a polymeric matrix. In some embodiments, the controlled volumes can be droplets, and each droplet may be independently formed and placed on a surface. Each droplet may independently include pure agent, a combination of agents, pure polymer, a combination of polymers, or a combination thereof. Likewise, the agents may be independently selected for each droplet.

The term “thickness” can refer to the distance between opposite surfaces of a polymeric matrix that is used in the production of a medical device or coating. The thickness can refer to that of a single layer, a single layer within a combination of layers, or a combination layers.

In some embodiments, the thickness of a polymeric matrix can be the thickness of a component within the structure of a medical device, such as, for example, the thickness of a strut within a stent. In other embodiments, the thickness of a polymeric matrix can be the thickness of a layer of coating applied to a medical device. In other embodiments, the thickness of a polymeric matrix can be the thickness of a combination of layers applied as a coating for a medical device. In many embodiments, the thickness of a polymeric matrix can range from about 0.1 nm to about 1.0 cm, from about 0.1 nm to about 1.0 mm, from about 0.1 nm to about 100 μm, from about 0.1 nm to about 1 μm, from about 0.1 nm to about 100 nm, from about 0.1 nm to about 10 nm, from about 10 nm to about 100 nm, from about 10 μm to about 50 μm, from about 50 μm to about 100 μm, or any range therein. In other embodiments, the thickness of a polymeric matrix can range from about 1 μm to about 10 μm, which can be found, for example, in some of the current drug-eluting stent (DES) systems. In other embodiments, the thickness of the polymeric matrices can be regionally distributed throughout a device to create a variation in thicknesses such as, for example, the variation in thicknesses that can be found in an abluminally coated DES stent.

In each of the embodiments, the term “layer” describes a thickness of a polymeric matrix within which an agent must pass through to be released into a subject. This term can refer, for example, to any individual polymeric matrix that may be used to form a medical device or a coating for a medical device. A layer can include, but is not limited to, polymeric material from a single-pass application or multiple-pass application, where a “pass” can be any single process step, or combination of steps, used to apply a material such as, for example, a pass of a spray coating device, a pass of an electrostatic coating device, a pass of a controlled-volume ejector, a dipping, an extrusion, a mold, a single dip in a layered manufacturing process, or a combination thereof. In general, a pass includes any single process step known to one of skill in the art that can be used to apply materials in the formation of a medical device or coating using a composition comprising a polymeric material. A layer can consist of a single pass or multiple passes. In some embodiments, the coating can be applied to an entire medical device or select regions of the medical device.

In some embodiments, the IC profile can be based primarily on the concentration gradient across a single layer. In these embodiments, the single layer may have a concentration gradient based on one or more agents that are dissolved in a polymer matrix and/or one or more agents that are in a dispersed phase within a polymer matrix.

In other embodiments, the IC profile across the polymeric matrix can be developed using a combination of layers, wherein each layer within the combination of layers may or may not include a controlled IC profile. In these embodiments, each layer within the combination of layers may have a concentration gradient based on one or more agents that are dissolved in a polymer matrix and/or one or more agents that are in a dispersed phase within a polymer matrix.

In other embodiments, the IC profile across the polymeric matrix can be developed using a combination of layers, wherein at least one of which contains a controlled IC profile, and the combination of layers provides an overall controlled IC profile. In these embodiments, each layer within the combination of layers may have a concentration gradient based on one or more agents that are dissolved in a polymer matrix and/or one or more agents that are in a dispersed phase within a polymer matrix.

Formation of Initial Concentration Gradient Profiles

There are many ways that an initial concentration profile can be formed through selection of material and process parameters. The material parameters include, but are not limited to, the selection of the polymer and/or polymer combinations, the selection of the agent and/or agent combinations, the selection of the polymer/agent combinations, and the selection of the solvent and/or solvent combinations used to combine the materials for application. The scope of the present invention includes, but is not limited to, the following materials and processes:

The Agent-Containing Compositions

The agent-containing compositions of the present invention include any combination of polymers, copolymers and agents. Compositions that are selected for an in vivo use should meet particular requirements with regard to physical, mechanical, chemical, and biological properties of the compositions. An example of a physical property that can affect the performance of a biodegradable composition in vivo is water uptake. An example of a mechanical property that can affect the performance of a composition in vivo is the ability of the composition to withstand stresses that can cause mechanical failure of the composition such as, for example, cracking, flaking, peeling, and fracturing. An example of a chemical property that can affect performance of a biodegradable composition in vivo is the rate of absorption of the composition by a subject. An example of a biological property that can affect performance of a composition in vivo is the bioactive and/or biobeneficial nature of the composition, both of which are described below. The terms “subject” and “patient” can be used interchangeably and refer to an animal such as a mammal including, but not limited to, non-primates such as, for example, a cow, pig, horse, cat, dog, rat, and mouse; and primates such as, for example, a monkey or a human.

While not intending to be bound by any theory or mechanism of action, water uptake by a composition can be an important characteristic in the design of a composition. Water can act as a plasticizer for modifying the mechanical properties of the composition. Control of water uptake can also provide some control over the hydrolysis of a coating and thus can provide control over the degradation rate, absorption rate, and the agent release rate of a medical article or coating in vivo. In some embodiments, an increase in hydrolysis can also increase the release rate of an agent by creating channels within a medical article or coating that can serve as transport pathways for diffusion of the agents from the composition within a subject.

The compositions of the present invention can be used in some embodiments to form medical devices and coatings that include a combination of agents, wherein each of the agents (i) can be incorporated in the device or coating without cross-contamination from the other agents; (ii) can perform its function substantially free from interference from the other agents, (ii) can be incorporated in the device or coating such that the agent has a predetermined release rate and absorption rate; and (iv) can be combined with other agents that are bioactive, biobeneficial, diagnostic, and/or control a physical property or a mechanical property of a medical device.

The terms “combine,” “combined,” “combining,” and “combination” all refer to a relationship between components of a composition and include blends, mixtures, linkages, and combinations thereof, of components that form the compositions. The linkages can be connections that are physical, chemical, or a combination thereof. Examples of physical connections include, but are not limited to, an interlinking of components that can occur, for example, in interpenetrating networks and chain entanglement. Examples of chemical connections include, but are not limited to, covalent and non-covalent bonds. Covalent bonds include, but are not limited to, simple covalent bonds and coordinate bonds. Non-covalent bonds include, but are not limited to, ionic bonds, and inter-molecular attractions such as, for example, hydrogen bonds and attractions created by induced and permanent dipole-dipole interactions. All of these types of combinations can have a variable effect on the measured diffusion coefficient.

A polymeric matrix can comprise polymers that are biodegradable, which can be due to the labile nature of chemical functionalities within the polymer network such as, for example, ester groups that can be present between chemical moieties. Accordingly, these compositions can be designed such that they can be broken down, absorbed, resorbed and eliminated by a mammal. The compositions of the present invention can be used, for example, to form medical articles and coatings. The polymers used in the present invention may include, but are not limited to, condensation copolymers, and should be chosen according to a desired performance parameter of a product that will be formed from the composition. Such performance parameters may include, for example, the toughness of a medical device or coating, the capacity for the loading concentration of an agent, and the rate of biodegradation and elimination of the composition from a subject. If the other polymers in a composition are non-biodegradable, they should be sized to produce polymer fragments that can clear from the subject following biodegradation of the composition.

For the purposes of the present invention, a polymer or coating is “biodegradable” when it is capable of being completely or substantially degraded or eroded when exposed to an in vivo environment or a representative in vitro. A polymer or coating is capable of being degraded or eroded when it can be gradually broken-down, resorbed, absorbed and/or eliminated by, for example, hydrolysis, enzymolysis, oxidation, metabolic processes, bulk or surface erosion, and the like within a subject. It should be appreciated that traces or residue of polymer may remain on the device, near the site of the device, or near the site of a biodegradable device, following biodegradation. The terms “bioabsorbable” and “biodegradable” are used interchangeably in this application.

In most embodiments, the polymers that can be used include natural or synthetic polymers; homopolymers and copolymers, such as, for example, copolymers that are random, alternating, block, graft, and/or crosslinked; or any combination and/or blend thereof. The copolymers include polymers with more than two different types of repeating units such as, for example, terpolymers.

In some embodiments, the number average molecular weight of the polymer fragments should be at or below about 40,000 Daltons, or any range therein. In other embodiments, the molecular weight of the fragments range from about 300 Daltons to about 40,000 Daltons, from about 8,000 Daltons to about 30,000 Daltons, from about 10,000 Daltons to about 20,000 Daltons, or any range therein. The molecular weights are taught herein as a number average molecular weight.

Examples of polymers that can be combined with the agents of the present invention include, but are not limited to, poly(acrylates) such as poly(butyl methacrylate), poly(ethyl methacrylate), poly(hydroxyl ethyl methacrylate), poly(ethyl methacrylate-co-butyl methacrylate), copolymers of ethylene-methyl methacrylate; poly (2-acrylamido-2-methylpropane sulfonic acid), and polymers and copolymers of aminopropyl methacrylamide; poly(cyanoacrylates); poly(carboxylic acids); poly(vinyl alcohols); poly(maleic anhydride) and copolymers of maleic anhydride; fluorinated polymers or copolymers such as poly(vinylidene fluoride), poly(vinylidene fluoride-co-hexafluoro propene), poly(tetrafluoroethylene), and expanded poly(tetrafluoroethylene); poly(sulfone); poly(N-vinyl pyrrolidone); poly(aminocarbonates); poly(iminocarbonates); poly(anhydride-co-imides), poly(hydroxyvalerate); poly(L-lactic acid); poly(L-lactide); poly(caprolactones); poly(lactide-co-glycolide); poly(hydroxybutyrates); poly(hydroxybutyrate-co-valerate); poly(dioxanones); poly(orthoesters); poly(anhydrides); poly(glycolic acid); poly(glycolide); poly(D,L-lactic acid); poly(D,L-lactide); poly(glycolic acid-co-trimethylene carbonate); poly(phosphoesters); poly(phosphoester urethane); poly(trimethylene carbonate); poly(iminocarbonate); poly(ethylene); poly(propylene) co-poly(ether-esters) such as, for example, poly(dioxanone) and poly(ethylene oxide)/poly(lactic acid); poly(anhydrides), poly(alkylene oxalates); poly(phosphazenes); poly(urethanes); silicones; poly(esters; poly(olefins); copolymers of poly(isobutylene); copolymers of ethylene-alphaolefin; vinyl halide polymers and copolymers such as poly(vinyl chloride); poly(vinyl ethers) such as poly(vinyl methyl ether); poly(vinylidene halides) such as, for example, poly(vinylidene chloride); poly(acrylonitrile); poly(vinyl ketones); poly(vinyl aromatics) such as poly(styrene); poly(vinyl esters) such as poly(vinyl acetate); copolymers of vinyl monomers and olefins such as poly(ethylene-co-vinyl alcohol) (EVAL), copolymers of acrylonitrile-styrene, ABS resins, and copolymers of ethylene-vinyl acetate; poly(amides) such as Nylon 66 and poly(caprolactam); alkyd resins; poly(carbonates); poly(oxymethylenes); poly(imides); poly(ester amides); poly(ethers) including poly(alkylene glycols) such as, for example, poly(ethylene glycol) and poly(propylene glycol); epoxy resins; polyurethanes; rayon; rayon-triacetate; biomolecules such as, for example, fibrin, fibrinogen, starch, poly(amino acids); peptides, proteins, gelatin, chondroitin sulfate, dermatan sulfate (a copolymer of D-glucuronic acid or L-iduronic acid and N-acetyl-D-galactosamine), collagen, hyaluronic acid, and glycosaminoglycans; other polysaccharides such as, for example, poly(N-acetylglucosamine), chitin, chitosan, cellulose, cellulose acetate, cellulose butyrate, cellulose acetate butyrate, cellophane, cellulose nitrate, cellulose propionate, cellulose ethers, and carboxymethylcellulose; and derivatives, analogs, homologues, congeners, salts, copolymers and combinations thereof. In some embodiments, the polymers are selected such that they specifically exclude any one or any combination of these polymers.

In some embodiments, the polymers can be biodegradable. Examples of biodegradable polymers include, but are not limited to, polymers having repeating units such as, for example, an α-hydroxycarboxylic acid, a cyclic diester of an α-hydroxycarboxylic acid, a dioxanone, a lactone, a cyclic carbonate, a cyclic oxalate, an epoxide, a glycol, an anhydride, a lactic acid, a glycolic acid, a lactide, a glycolide, an ethylene oxide, an ethylene glycol, or combinations thereof. In other embodiments, the biodegradable polymers include, but are not limited to, polyesters, poly(ester amides); poly(hydroxyalkanoates) (PHA), amino acids; PEG and/or alcohol groups, polycaprolactones, poly(D-lactide), poly(L-lactide), poly(D,L-lactide), poly(meso-lactide), poly(L-lactide-co-meso-lactide), poly(D-lactide-co-meso-lactide), poly(D,L-lactide-co-meso-lactide), poly(D,L-lactide-co-PEG) block copolymers, poly(D,L-lactide-co-trimethylene carbonate), polyglycolides, poly(lactide-co-glycolide), polydioxanones, polyorthoesters, polyanhydrides, poly(glycolic acid-co-trimethylene carbonate), polyphosphoesters, polyphosphoester urethanes, poly(amino acids), polycyanoacrylates, poly(trimethylene carbonate), poly(imino carbonate), polycarbonates, polyurethanes, copoly(ether-esters) (e.g. PEO/PLA), polyalkylene oxalates, polyphosphazenes, PHA-PEG, and any derivatives, analogs, homologues, salts, copolymers and combinations thereof.

In other embodiments, the polymers can be poly(glycerol sebacate); tyrosine-derived polycarbonates containing desaminotyrosyl-tyrosine alkyl esters such as, for example, desaminotyrosyl-tyrosine ethyl ester (poly(DTE carbonate)); and any derivatives, analogs, homologues, salts, copolymers and combinations thereof. In some embodiments, the polymers are selected such that they specifically exclude any one or any combination of any of the polymers taught herein.

In some embodiments, the polymers can be chemically connected to the agents by covalent bonds. In other embodiments, the polymers can be chemically connected to the agents by non-covalent bonds such as, for example, by ionic bonds, inter-molecular attractions, or a combination thereof. In other embodiments, the polymers can be physically connected to the agents. In other embodiments, the polymers can be chemically and physically connected with the agents. Examples of ionic bonding can include, but are not limited to, ionic bonding of an anionic site to a cationic site between polymers. In some embodiments, an anionic site can be bound to a quaternary amine. Examples of inter-molecular attractions include, but are not limited to, hydrogen bonding such as, for example, the permanent dipole interactions between hydroxyl, amino, carboxyl, amide, and sulfhydryl groups, and combinations thereof. Examples of physical connections can include, but are not limited to, interpenetrating networks and chain entanglement. The polymers can also be blended or mixed with the agents.

The Agents

Biobeneficial and Bioactive Agents

A “bioactive agent” is a moiety that can be combined with a polymer and provides a therapeutic effect, a prophylactic effect, both a therapeutic and a prophylactic effect, or other biologically active effect within a subject. Moreover, the bioactive agents of the present invention may remain linked to a portion of the polymer or be released from the polymer. A “biobeneficial agent” is an agent that can be combined with a polymer and provide a biological benefit within a subject without necessarily being released from the polymer.

In one example, a biological benefit may be that the polymer or coating becomes non-thrombogenic, such that protein absorption is inhibited or prevented to avoid formation of a thromboembolism; promotes healing, such that endothelialization within a blood vessel is not exuberant but rather forms a healthy and functional endothelial layer; or is non-inflammatory, such that the biobeneficial agent acts as a biomimic to passively avoid attracting monocytes and neutrophils, which could lead to an event or cascade of events that create inflammation.

A “diagnostic agent” is a type of bioactive agent that can be used, for example, in diagnosing the presence, nature, or extent of a disease or medical condition in a subject. In one embodiment, a diagnostic agent can be any agent that may be used in connection with methods for imaging an internal region of a patient and/or diagnosing the presence or absence of a disease in a patient. Diagnostic agents include, for example, contrast agents for use in connection with ultrasound imaging, magnetic resonance imaging (MRI), nuclear magnetic resonance (NMR), computed tomography (CT), electron spin resonance (ESR), nuclear medical imaging, optical imaging, elastography, and radiofrequency (RF) and microwave lasers. Diagnostic agents may also include any other agents useful in facilitating diagnosis of a disease or other condition in a patient, whether or not imaging methodology is employed.

Examples of biobeneficial agents include, but are not limited to, many of the polymers listed above such as, for example, carboxymethylcellulose; poly(alkylene glycols) such as, for example, PEG; poly(N-vinyl pyrrolidone); poly(acrylamide methyl propane sulfonic acid); poly(styrene sulfonate); sulfonated polysaccharides such as, for example, sulfonated dextran; sulfated polysaccharides such as, for example, sulfated dextran and dermatan sulfate; and glycosaminoglycans such as, for example, hyaluronic acid and heparin; and any derivatives, analogs, homologues, congeners, salts, copolymers and combinations thereof. In some embodiments, the biobeneficial agents can be prohealing such as, for example, poly(ester amides), elastin, silk-elastin, collagen, atrial natriuretic peptide (ANP); and peptide sequences such as, for example, those comprising Arg-Gly-Asp (RGD). In other embodiments, the biobeneficial agents can be non-thrombotics such as, for example, thrombomodulin; and antimicrobials such as, for example, the organosilanes. It is to be appreciated that one skilled in the art should recognize that some of the groups, subgroups, and individual biobeneficial agents may not be used in some embodiments of the present invention.

Examples of heparin derivatives include, but are not limited to, earth metal salts of heparin such as, for example, sodium heparin, potassium heparin, lithium heparin, calcium heparin, magnesium heparin, and low molecular weight heparin. Other examples of heparin derivatives include, but are not limited to, heparin sulfate, heparinoids, heparin-based compounds and heparin derivatized with hydrophobic materials.

Examples of hyaluronic acid derivates include, but are not limited to, sulfated hyaluronic acid such as, for example, O-sulphated or N-sulphated derivatives; esters of hyaluronic acid wherein the esters can be aliphatic, aromatic, arylaliphatic, cycloaliphatic, heterocyclic or a combination thereof; crosslinked esters of hyaluronic acid wherein the crosslinks can be formed with hydroxyl groups of a polysaccharide chain; crosslinked esters of hyaluronic acid wherein the crosslinks can be formed with polyalcohols that are aliphatic, aromatic, arylaliphatic, cycloaliphatic, heterocyclic, or a combination thereof; hemiesters of succinic acid or heavy metal salts thereof; quaternary ammonium salts of hyaluronic acid or derivatives such as, for example, the O-sulphated or N-sulphated derivatives.

Examples of poly(alkylene glycols) include, but are not limited to, PEG, mPEG, poly(ethylene oxide), polypropylene glycol)(PPG), poly(tetramethylene glycol), and any derivatives, analogs, homologues, congeners, salts, copolymers and combinations thereof. In some embodiments, the poly(alkylene glycol) is PEG. In other embodiments, the poly(alkylene glycol) is mPEG. In other embodiments, the poly(alkylene glycol) is poly(ethylene glycol-co-hydroxybutyrate).

The copolymers that may be used as biobeneficial agents include, but are not limited to, any derivatives, analogs, homologues, congeners, salts, copolymers and combinations of the foregoing examples of agents. Examples of copolymers that may be used as biobeneficial agents in the present invention include, but are not limited to, dermatan sulfate, which is a copolymer of D-glucuronic acid or L-iduronic acid and N-acetyl-D-galactosamine; poly(ethylene oxide-co-propylene oxide); copolymers of PEG and hyaluronic acid; copolymers of PEG and heparin; copolymers of PEG and hirudin; graft copolymers of poly(L-lysine) and PEG; copolymers of PEG and a poly(hydroxyalkanoate) such as, for example, poly(ethylene glycol-co-hydroxybutyrate); and, any derivatives, analogs, congeners, salts, or combinations thereof. In some embodiments, the copolymer that may be used as a biobeneficial agent can be a copolymer of PEG and hyaluronic acid, a copolymer of PEG and hirudin, and any derivative, analog, congener, salt, copolymer or combination thereof. In other embodiments, the copolymer that may be used as a biobeneficial agent is a copolymer of PEG and a poly(hydroxyalkanoate) such as, for example, poly(hydroxybutyrate); and any derivative, analog, congener, salt, copolymer or combination thereof.

The bioactive agents can be any moiety capable of contributing to a therapeutic effect, a prophylactic effect, both a therapeutic and prophylactic effect, or other biologically active effect in a mammal. The agent can also have diagnostic properties. The bioactive agents include, but are not limited to, small molecules, nucleotides, oligonucleotides, polynucleotides, amino acids, oligopeptides, polypeptides, and proteins. In one embodiment, the bioactive agent inhibits the activity of vascular smooth muscle cells. In another embodiment, the bioactive agent can be used to control migration or proliferation of smooth muscle cells to inhibit restenosis. In another embodiment, the bioactive agent can be used in the prevention and/or treatment of restenosis and/or vulnerable plaque. In some embodiments, the term “treatment” includes, but is not limited to, the mitigation, diagnosis, ameliorization of the symptoms, or a combination thereof, of a disease.

Bioactive agents include, but are not limited to, antiproliferatives, antineoplastics, antimitotics, anti-inflammatories, antiplatelets, anticoagulants, antifibrins, antithrombins, antibiotics, antiallergics, antioxidants, and any prodrugs, metabolites, analogs, homologues, congeners, derivatives, salts and combinations thereof. It is to be appreciated that one skilled in the art should recognize that some of the groups, subgroups, and individual bioactive agents may not be used in some embodiments of the present invention.

Antiproliferatives include, for example, actinomycin D, actinomycin IV, actinomycin I₁, actinomycin X₁, actinomycin C₁, dactinomycin (COSMEGEN®, Merck & Co., Inc.), imatinib mesylate, and any prodrugs, metabolites, analogs, homologues, congeners, derivatives, salts and combinations thereof. Antineoplastics or antimitotics include, for example, paclitaxel (TAXOL®, Bristol-Myers Squibb Co.), docetaxel (TAXOTERE®, Aventis S.A.), midostaurin, methotrexate, azathioprine, vincristine, vinblastine, fluorouracil, doxorubicin hydrochloride (ADRIAMYCIN®, Pfizer, Inc.) and mitomycin (MUTAMYCIN®, Bristol-Myers Squibb Co.), midostaurin, and any prodrugs, metabolites, analogs, homologues, congeners, derivatives, salts and combinations thereof.

Antiplatelets, anticoagulants, antifibrin, and antithrombins include, for example, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors (ANGIOMAX®, Biogen, Inc.), and any prodrugs, metabolites, analogs, homologues, congeners, derivatives, salts and combinations thereof.

Cytostatic or antiproliferative agents include, for example, angiopeptin, angiotensin converting enzyme inhibitors such as captopril (CAPOTEN® and CAPOZIDE®, Bristol-Myers Squibb Co.), cilazapril or lisinopril (PRINIVIL® and PRINZIDE®, Merck & Co., Inc.); calcium channel blockers such as nifedipine; colchicines; fibroblast growth factor (FGF) antagonists, fish oil (omega 3-fatty acid); histamine antagonists; lovastatin (MEVACOR®, Merck & Co., Inc.); monoclonal antibodies including, but not limited to, antibodies specific for Platelet-Derived Growth Factor (PDGF) receptors; nitroprusside; phosphodiesterase inhibitors; prostaglandin inhibitors; suramin; serotonin blockers; steroids; thioprotease inhibitors; PDGF antagonists including, but not limited to, triazolopyrimidine; and nitric oxide; imatinib mesylate; and any prodrugs, metabolites, analogs, homologues, congeners, derivatives, salts and combinations thereof. Antiallergic agents include, but are not limited to, pemirolast potassium (ALAMAST®, Santen, Inc.), and any prodrugs, metabolites, analogs, homologues, congeners, derivatives, salts and combinations thereof.

Other bioactive agents useful in the present invention include, but are not limited to, free radical scavengers; nitric oxide donors; rapamycin; methyl rapamycin; 42-Epi-(tetrazoylyl)rapamycin (ABT-578); 40-O-(2-hydroxy)ethyl-rapamycin (everolimus); tacrolimus; pimecrolimus; 40-O-(3-hydroxy)propyl-rapamycin; 40-O-[2-(2-hydroxy)ethoxy]ethyl-rapamycin; tetrazole containing rapamycin analogs such as those described in U.S. Pat. No. 6,329,386; estradiol; clobetasol; idoxifen; tazarotene; alpha-interferon; host cells such as epithelial cells; genetically engineered epithelial cells; dexamethasone; and, any prodrugs, metabolites, analogs, homologues, congeners, derivatives, salts and combinations thereof.

Free radical scavengers include, but are not limited to, 2,2′,6,6′-tetramethyl-1-piperinyloxy, free radical (TEMPO); 4-amino-2,2′,6,6′-tetramethyl-1-piperinyloxy, free radical (4-amino-TEMPO); 4-hydroxy-2,2′,6,6′-tetramethyl-piperidene-1-oxy, free radical (TEMPOL), 2,2′,3,4,5,5′-hexamethyl-3-imidazolinium-1-yloxy methyl sulfate, free radical; 16-doxyl-stearic acid, free radical; superoxide dismutase mimic (SODm) and any analogs, homologues, congeners, derivatives, salts and combinations thereof. Nitric oxide donors include, but are not limited to, S-nitrosothiols, nitrites, N-oxo-N-nitrosamines, substrates of nitric oxide synthase, diazenium diolates such as spermine diazenium diolate and any analogs, homologues, congeners, derivatives, salts and combinations thereof.

Examples of diagnostic agents include radioopaque materials and include, but are not limited to, materials comprising iodine or iodine-derivatives such as, for example, iohexal and iopamidol, which are detectable by x-rays. Other diagnostic agents such as, for example, radioisotopes, are detectable by tracing radioactive emissions. Other diagnostic agents may include those that are detectable by magnetic resonance imaging (MRI), ultrasound and other imaging procedures such as, for example, fluorescence and positron emission tomagraphy (PET). Examples of agents detectable by MRI are paramagnetic agents, which include, but are not limited to, gadolinium chelated compounds. Examples of agents detectable by ultrasound include, but are not limited to, perflexane. Examples of fluorescence agents include, but are not limited to, indocyanine green. Examples of agents used in diagnostic PET include, but are not limited to, fluorodeoxyglucose, sodium fluoride, methionine, choline, deoxyglucose, butanol, raclopride, spiperone, bromospiperone, carfentanil, and flumazenil.

In some embodiments, a combination of agents can be applied, as taught herein, within predetermined IC profiles within a medical device, on a medical device, or positioned within a controlled volume at a predetermined region on the device or within a coating on the device. In some embodiments, the agent combination includes everolimus and clobetasol. In other embodiments, the agent combination includes tacrolimus and rapamycin. In other embodiments, the agent combination includes tacrolimus and everolimus. In other embodiments, the agent combination can include rapamycin and paclitaxel. In other embodiments, the agent combination can include an anti-inflammatory such as, for example, a corticosteroid and an antiproliferative such as, for example, everolimus. In some embodiments, the agent combinations can provide synergistic effects for preventing or inhibiting conditions such as, for example, restenosis that may occur through use of a stent.

Plasticizing Agents

The terms “plasticizer” and “plasticizing agent” can be used interchangeably in the present invention, and refer to any agent, including any agent described above, where the agent can be added to a polymeric composition to modify the mechanical properties of the composition or a product formed from the composition. Plasticizers can be added, for example, to reduce crystallinity, lower the glass-transition temperature (T_{g}), or reduce the intermolecular forces between polymers, with design goals that may include, but are not limited to, enhancing mobility between polymer chains in the composition. The mechanical properties that are modified include, but are not limited to, Young's modulus, impact resistance (toughness), tensile strength, and tear strength. Impact resistance, or “toughness,” is a measure of energy absorbed during fracture of a polymer sample of standard dimensions and geometry when subjected to very rapid impact loading. Toughness can be measured using Charpy and Izod impact tests to assess the brittleness of a material.

A plasticizer can be monomeric, polymeric, co-polymeric, or a combination thereof, and can be combined with a polymeric composition in the same manner as described above for the biobeneficial and bioactive agents. Plasticization and solubility are analogous in the sense that selecting a plasticizer involves considerations similar to selecting a solvent such as, for example, polarity. Furthermore, plasticization can also be provided through covalent bonding by changing the molecular structure of the polymer through copolymerization.

Examples of plasticizing agents include, but are not limited to, low molecular weight polymers such as, for example, single-block polymers, multi-block copolymers, and other copolymers such as graft copolymers; oligomers such as ethyl-terminated oligomers of lactic acid; small organic molecules; hydrogen bond forming organic compounds with and without hydroxyl groups; polyols such as low molecular weight polyols having aliphatic hydroxyls; alkanols such as butanols, pentanols and hexanols; sugar alcohols and anhydrides of sugar alcohols; polyethers such as poly(alkylene glycols); esters such as citrates, phthalates, sebacates and adipates; polyesters; aliphatic acids; proteins such as animal proteins and vegetable proteins; oils such as, for example, the vegetable oils and animal oils; silicones; acetylated monoglycerides; amides; acetamides; sulfoxides; sulfones; pyrrolidones; oxa acids; diglycolic acids; and any analogs, derivatives, copolymers and combinations thereof.

In some embodiments, the plasticizers include, but are not limited to other polyols such as, for example, caprolactone diol, caprolactone triol, sorbitol, erythritol, glucidol, mannitol, sorbitol, sucrose, and trimethylol propane. In other embodiments, the plasticizers include, but are not limited to, glycols such as, for example, ethylene glycol, diethylene glycol, triethylene glycol, tetraethylene glycol, propylene glycol, butylene glycol, 1,2-butylene glycol, 2,3-butylene glycol, styrene glycol, pentamethylene glycol, hexamethylene glycol; glycol-ethers such as, for example, monopropylene glycol monoisopropyl ether, propylene glycol monoethyl ether, ethylene glycol monoethyl ether, and diethylene glycol monoethyl ether; and any analogs, derivatives, copolymers and combinations thereof.

In other embodiments, the plasticizers include, but are not limited to esters such as glycol esters such as, for example, diethylene glycol dibenzoate, dipropylene glycol dibenzoate, triethylene glycol caprate-caprylate; monostearates such as, for example, glycerol monostearate; citrate esters; organic acid esters; aromatic carboxylic esters; aliphatic dicarboxylic esters; fatty acid esters such as, for example, stearic, oleic, myristic, palmitic, and sebacic acid esters; triacetin; poly(esters) such as, for example, phthalate polyesters, adipate polyesters, glutate polyesters, phthalates such as, for example, dialkyl phthalates, dimethyl phthalate, diethyl phthalate, isopropyl phthalate, dibutyl phthalate, dihexyl phthalate, dioctyl phthalate, diisononyl phthalate, and diisodecyl phthalate; sebacates such as, for example, alkyl sebacates, dimethyl sebacate, dibutyl sebacate; hydroxyl-esters such as, for example, lactate, alkyl lactates, ethyl lactate, butyl lactate, allyl glycolate, ethyl glycolate, and glycerol monostearate; citrates such as, for example, alkyl acetyl citrates, triethyl acetyl citrate, tributyl acetyl citrate, trihexyl acetyl citrate, alkyl citrates, triethyl citrate, and tributyl citrate; esters of castor oil such as, for example, methyl ricinolate; aromatic carboxylic esters such as, for example, trimellitic esters, benzoic esters, and terephthalic esters; aliphatic dicarboxylic esters such as, for example, dialkyl adipates, alkyl allylether diester adipates, dibutoxyethoxyethyl adipate, diisobutyl adipate, sebacic esters, azelaic esters, citric esters, and tartaric esters; and fatty acid esters such as, for example, glycerol, mono- di- or triacetate, and sodium diethyl sulfosuccinate; and any analogs, derivatives, copolymers and combinations thereof.

In other embodiments, the plasticizers include, but are not limited to ethers and polyethers such as, for example, poly(alkylene glycols) such as poly(ethylene glycols) (PEG), polypropylene glycols), and poly(ethylene/propylene glycols); low molecular weight poly(ethylene glycols) such as, for example, PEG 400 and PEG 6000; PEG derivatives such as, for example, methoxy poly(ethylene glycol) (mPEG); and ester-ethers such as, for example, diethylene glycol dibenzoate, dipropylene glycol dibenzoate, and triethylene glycol caprate-caprylate; and any analogs, derivatives, copolymers and combinations thereof.

In other embodiments, the plasticizers include, but are not limited to, amides such as, for example, oleic amide, erucic amide, and palmitic amide; alkyl acetamides such as, for example, dimethyl acetamide and dimethyl formamide; sulfoxides such as for example, dimethyl sulfoxide; pyrrolidones such as, for example, n-methyl pyrrolidone; sulfones such as, for example, tetramethylene sulfone; acids such as, for example, oxa monoacids, oxa diacids such as 3,6,9-trioxaundecanedioic acid, polyoxa diacids, ethyl ester of acetylated citric acid, butyl ester of acetylated citric acid, capryl ester of acetylated citric acid, and diglycolic acids such as dimethylol propionic acid; and any analogs, derivatives, copolymers and combinations thereof.

In other embodiments, the plasticizers can be vegetable oils including, but not limited to, epoxidized soybean oil; linseed oil; castor oil; coconut oil; fractionated coconut oil; epoxidized tallates; and esters of fatty acids such as stearic, oleic, myristic, palmitic, and sebacic acid. In other embodiments, the plasticizers can be essential oils including, but not limited to, angelica oil, anise oil, arnica oil, aurantii aetheroleum, valerian oil, basilici aetheroleum, bergamot oil, savory oil, bucco aetheroleum, camphor, cardamomi aetheroleum, cassia oil, chenopodium oil, chrysanthemum oil, cinae aetheroleum, citronella oil, lemon oil, citrus oil, costus oil, curcuma oil, carlina oil, elemi oil, tarragon oil, eucalyptus oil, fennel oil, pine needle oil, pine oil, filicis, aetheroleum, galbanum oil, gaultheriae aetheroleum, geranium oil, guaiac wood oil, hazelwort oil, iris oil, hypericum oil, calamus oil, camomile oil, fir needle oil, garlic oil, coriander oil, carraway oil, lauri aetheroleum, lavender oil, lemon grass oil, lovage oil, bay oil, lupuli strobuli aetheroleum, mace oil, marjoram oil, mandarine oil, melissa oil, menthol, millefolii aetheroleum, mint oil, clary oil, nutmeg oil, spikenard oil, clove oil, neroli oil, niaouli, olibanum oil, ononidis aetheroleum, opopranax oil, orange oil, oregano oil, orthosiphon oil, patchouli oil, parsley oil, petit-grain oil, peppermint oil, tansy oil, rosewood oil, rose oil, rosemary oil, rue oil, sabinae aetheroleum, saffron oil, sage oil, sandalwood oil, sassafras oil, celery oil, mustard oil, serphylli aetheroleum, immortelle oil, fir oil, teatree oil, terpentine oil, thyme oil, juniper oil, frankincense oil, hyssop oil, cedar wood oil, cinnamon oil, and cypress oil; and other oils such as, for example, fish oil; and, any analogs, derivatives, copolymers and combinations thereof.

The molecular weights of the plasticizers can vary. In some embodiments, the molecular weights of the plasticizers range from about 10 Daltons to about 50,000 Daltons; from about 25 Daltons to about 25,000 Daltons; from about 50 Daltons to about 10,000 Daltons; from about 100 Daltons to about 5,000 Daltons; from about 200 Daltons to about 2500 Daltons; from about 400 Daltons to about 1250 Daltons; and any range therein. In other embodiments, the molecular weights of the plasticizers range from about 400 Daltons to about 4000 Daltons; from about 300 Daltons to about 3000 Daltons; from about 200 Daltons to about 2000 Daltons; from about 100 Daltons to about 1000 Daltons; from about 50 Daltons to about 5000 Daltons; and any range therein. The molecular weights are taught herein as a number average molecular weight.

The amount of plasticizer used in the present invention, can range from about 0.001% to about 70%; from about 0.01% to about 60%; from about 0.1% to about 50%; from about 0.1% to about 40%; from about 0.1% to about 30%; from about 0.1% to about 25%; from about 0.1% to about 20%; from about 0.1% to about 10%; from about 0.4% to about 40%; from about 0.6% to about 30%; from about 0.75% to about 25%; from about 1.0% to about 20%; and any range therein, as a weight percentage based on the total weight of the polymer and agent or combination of agents.

It should be appreciated that any one or any combination of the plasticizers described above can be used in the present invention. For example, the plasticizers can be combined to obtain the desired function. In some embodiments, a secondary plasticizer is combined with a primary plasticizer in an amount that ranges from about 0.001% to about 20%; from about 0.01% to about 15%; from about 0.05% to about 10%; from about 0.75% to about 7.5%; from about 1.0% to about 5%, or any range therein, as a weight percentage based on the total weight of the polymer any agent or combination of agents.

It should also be appreciated that the plasticizers can be combined with other active agents to obtain other desired functions such as, for example, an added therapeutic, prophylactic, and/or diagnostic function. In some embodiments, the plasticizers can be linked to other agents through ether, amide, ester, orthoester, anhydride, ketal, acetal, carbonate, and all-aromatic carbonate linkages, which are discussed in more detail below.

In some embodiments, the agents can be chemically connected to a polymer by covalent bonds. In other embodiments, the agents can be chemically connected to a polymer by non-covalent bonds such as, for example, by ionic bonds, inter-molecular attractions, or a combination thereof. In other embodiments, the agents can be physically connected to a polymer. In other embodiments, the agents can be chemically and physically connected with a polymer.

Examples of ionic bonding can include, but are not limited to, ionic bonding of an anionic agent to a cationic site on a polymer or a cationic agent to an anionic site on a polymer. In some embodiments, an anionic agent can be bound to a quaternary amine on a polymer. In other embodiments, an agent with a quaternary amine can be bound to an anionic site on a polymer. Examples of inter-molecular attractions include, but are not limited to, hydrogen bonding such as, for example, the permanent dipole interactions between hydroxyl, amino, carboxyl, and sulfhydryl groups, and combinations thereof. Examples of physical connections can include, but are not limited to, interpenetrating networks and chain entanglement. The agents can also be blended or mixed with the compositions.

In some embodiments, the agents have a reactive group that can be used to link the agents to the polymer. Examples of reactive groups include, but are not limited to, hydroxyl, acyl, amino, amido, and sulfhydryl groups. In some embodiments, the agents can be released or can separate from the polymer composition. In other embodiments, the agents can be biobeneficial, bioactive, diagnostic, plasticizing, or have a combination of these characteristics.

In some embodiments, the molecular weight of an agent should be at or below about 40,000 Daltons, or any range therein, to ensure elimination of the agent from a mammal. In one embodiment, the molecular weight of the agent ranges from about 300 Daltons to about 40,000 Daltons, from about 8,000 Daltons to about 30,000 Daltons, from about 10,000 Daltons to about 20,000 Daltons, or any range therein. If upon release, the biobeneficial agent is rapidly broken down in the body, then the molecular weight of the agent could be greater than about 40,000 Daltons without compromising patient safety. The molecular weights as taught herein are a number average molecular weight.

It should also be appreciated that the agents of the present invention can have properties that are biobeneficial, bioactive, diagnostic, plasticizing or a combination thereof. For example, classification of an agent as a biobeneficial agent does not preclude the use of that agent as a bioactive agent, diagnostic agent and/or plasticizing agent. Likewise, classification of an agent as a bioactive agent does not preclude the use of that agent as a diagnostic agent, biobeneficial agent and/or plasticizing agent. Furthermore, classification of an agent as a plasticizing agent does not preclude the use of that agent as a biobeneficial agent, bioactive agent, and/or diagnostic agent. It should also be appreciated that any of the foregoing agents can be combined with the compositions such as, for example, in the form of a medical device or a coating for a medical device. By way of a non-limiting example, a stent coated with the compositions of the invention can contain paclitaxel, docetaxel, rapamycin, methyl rapamycin, ABT-578, everolimus, clobetasol, pimecrolimus, imatinib mesylate, medostaurin, or combinations thereof.

Concentrations of Agents

The agents of the present invention can be added in combination to obtain other desired functions of the polymeric compositions. The amounts of the agents that compose the polymeric compositions vary according to a variety of factors including, but not limited to, the biological activity of the agent; the age, body weight, response, or the past medical history of the subject; the type of atherosclerotic disease; the presence of systemic diseases such as, for example, diabetes; the pharmacokinetic and pharmacodynamic effects of the agents or combination of agents; and the design of the compositions for sustained release of the agents. Factors such as these are routinely considered by one of skill in the art when administering an agent to a subject.

It is to be appreciated that the design of a composition for the sustained release of agents can be dependent on a variety of factors such as, for example, the therapeutic, prophylactic, ameliorative or diagnostic needs of a patient. In some embodiments, the agent can comprise an antiproliferative and should have a sustained release ranging from about 1 week to about 10 weeks, from about 2 weeks to about 8 weeks, from about 3 weeks to about 7 weeks, from about 4 weeks to about 6 weeks, and any range therein. In other embodiments, the agent can comprise an anti-inflammatory and should have a sustained release ranging from about 6 hours to about 3 weeks, from about 12 hours to about 2 weeks, from about 18 hours to about 10 days, from about 1 day to about 7 days, from about 2 days to about 6 days, or any range therein. In general, the sustained release should range from about 4 hours to about 12 weeks; alternatively, from about 6 hours to about 10 weeks; or from about 1 day to about 8 weeks.

Effective amounts, for example, may be extrapolated from in vitro or animal model systems. In some embodiments, the agent or combination of agents have a concentration that ranges from about 0.001% to about 75%; from about 0.01% to about 70%; from about 0.1% to about 60%; from about 0.25% to about 60%; from about 0.5% to about 50%; from about 0.75% to about 40%; from about 1.0% to about 30%; from about 2% to about 20%; and, any range therein, where the percentage is based on the total weight of the polymer and agent or combination of agents.

The formation of the medical devices and coatings of the present invention may require the selection and use of solvents to assist in creating and using the compositions of the present invention. Since many applications of the present invention include “casting” of the compositions, the solvents will be referred to as “casting solvents.” The casting solvent used to form medical devices or coatings may be chosen based on several criteria including, for example, its polarity, ability to hydrogen bond, molecular size, volatility, biocompatibility, reactivity and purity. Other physical characteristics of the casting solvent may also be taken into account including the solubility limit of the polymer in the casting solvent; the presence of oxygen and other gases in the casting solvent; the viscosity and vapor pressure of the combined casting solvent and polymer; the ability of the casting solvent to diffuse through adjacent materials, such as an underlying material; and the thermal stability of the casting solvent.

One of skill in the art has access to scientific literature and data regarding the solubility of a wide variety of polymers. Furthermore, one of skill in the art will appreciate that the choice of casting solvent may begin empirically by calculating the Gibb's free energy of dissolution using available thermodynamic data. Such calculations allow for a preliminary selection of potential solvents to test in a laboratory. It is recognized that process conditions can affect the chemical structure of the underlying materials and, thus, affect their solubility in a casting solvent. It is also recognized that the kinetics of dissolution are a factor to consider when selecting a casting solvent, because a slow dissolution of an underlying material, for example, may not affect the performance characteristics of a product where the product is produced relatively quickly.

Exemplary casting solvents for use in the present invention include, but are not limited to, DMAC, DMF, THF, cyclohexanone, xylene, toluene, acetone, i-propanol, methyl ethyl ketone, propylene glycol monomethyl ether, methyl butyl ketone, ethyl acetate, n-butyl acetate, and dioxane. Solvent mixtures can be used as well. Representative examples of the mixtures include, but are not limited to, DMAC and methanol (50:50 w/w); water, i-propanol, and DMAC (10:3:87 w/w); i-propanol and DMAC (80:20, 50:50, or 20:80 w/w); acetone and cyclohexanone (80:20, 50:50, or 20:80 w/w); acetone and xylene (50:50 w/w); acetone, xylene and FLUX REMOVER AMS® (93.7% 3,3-dichloro-1,1,1,2,2-pentafluoropropane and 1,3-dichloro-1,1,2,2,3-pentafluoropropane, and the balance is methanol with trace amounts of nitromethane; Tech Spray, Inc.) (10:40:50 w/w); and 1,1,2-trichloroethane and chloroform (80:20 w/w).

The process parameters include, but are not limited to, the selection of the process or combination of processes used to form a medical device or coating, in which the processes can include all of the steps from selection of the components of the composition and forming the composition to applying, forming, drying, and possibly annealing the composition in making a medical device or coating. The following methods are examples of methods that can be used in producing the medical devices and coatings of the present invention. These methods are not intended to be limiting for purposes of the present invention.

Forming a Medical Article

The agent can be localized as an IC profile in an implant during a process of forming the implant, and the localization can be beneficial for a variety of reasons such as, for example, use of less agent in select regions; use of a preferred agent in select regions such as, for example, an agent with desired potency or faster leaching rate; modification of mechanical properties of select regions of an implant; leaching of less agent for elimination by a subject; and combinations thereof. In some embodiments, there may be no agent in the regions outside of the high-strain regions in an implant. In other embodiments, there may be less agent in the regions outside of the high-strain regions in an implant. In embodiments where less agent is desired in the regions outside of the high-strain regions, the amount of agent in the regions outside of the high-strain regions can have 2%, 5%, 10%, 15%, 20%, 30%, 40%, 50%, 60%, 70%, 80%, 90%, 95%, or any range therein, less agent than the high-strain regions.

Processes for forming a medical article include, but are not limited to, casting, molding, coating, and combinations thereof. In some embodiments, the agent-containing compositions can be applied within the process in the form of a controlled volume, such as a droplet. In some embodiments, the implant is formed in a casting process, and the mechanical properties of the high-strain regions of the implant are controlled by concentrating the agent in the high-strain regions, by using different agents in the high-strain regions, by using agents only in the high-strain regions, or a combination thereof. Casting an implant involves pouring a liquid polymeric composition into a mold. In one embodiment, the localization of an agent in an implant during such casting can be obtained by varying the amount and/or type of agent in the polymeric composition during pouring as desired such that the agent becomes localized in the formed implant.

In other embodiments, the implant is formed in a molding process, which includes, but is not limited to, compression molding, extrusion molding, injection molding, and foam molding. The mechanical properties of the high-strain regions of the implant are controlled by concentrating the agent in the high-strain regions, by using different agents in the high-strain regions, by using agents only in the high-strain regions, or a combination thereof.

In compression molding, solid polymeric materials are added to a mold and pressure and heat are applied until the polymeric material conforms to the mold. The solid form may require additional processing to obtain the final product in a desired form. The solid polymeric materials can be in the form of particles that can vary in mean diameter from about 1 nm to about 1 cm, from about 1 nm to about 10 mm, from about 1 nm to about 1 mm, from about 1 nm to about 100 nm, or any range therein. In one embodiment, the localization of agents in an implant during such compression molding can be obtained by varying the amount and/or type of agent in the solid polymeric materials while adding the solid polymeric materials to the mold as desired such that the agent becomes localized in the formed implant.

In extrusion molding, solid polymeric materials are added to a continuous melt that is forced through a die and cooled to a solid form. The solid form may require additional processing to obtain the final product in a desired form. The solid polymeric materials can be in the form of particles that can vary in mean diameter from about 1 nm to about 1 cm, from about 1 nm to about 10 mm, from about 1 nm to about 1 mm, from about 1 nm to about 100 nm, or any range therein. In one embodiment, the localization of agent in an implant during such extrusion molding can be obtained by varying the amount and/or type of agent in the solid polymeric materials while adding the solid polymeric materials to the extrusion mold as desired such that the agent becomes localized in the formed implant.

In injection molding, solid polymeric materials are added to a heated cylinder, softened and forced into a mold under pressure to create a solid form. The solid form may require additional processing to obtain the final product in a desired form. The solid polymeric materials can be in the form of particles that can vary in mean diameter from about 1 nm to about 1 cm, from about 1 nm to about 10 mm, from about 1 nm to about 1 mm, from about 1 nm to about 100 nm, or any range therein. In one embodiment, the localization of agent in an implant during such injection molding can be obtained by varying the amount and/or type of agent in the solid polymeric materials while adding the solid polymeric materials to the injection mold as desired such that the agent becomes localized in the formed implant.

In foam molding, blowing agents are used to expand and mold solid polymeric materials into a desired form, and the solid polymeric materials can be expanded to a volume ranging from about two to about 50 times their original volume. The polymeric material can be pre-expanded using steam and air and then formed in a mold with additional steam; or mixed with a gas to form a polymer/gas mixture that is forced into a mold of lower pressure. The solid form may require additional processing to obtain the final product in a desired form. The solid polymeric materials can be in the form of particles that can vary in mean diameter from about 1 nm to about 1 cm, from about 1 nm to about 10 mm, from about 1 nm to about 1 mm, from about 1 nm to about 100 nm, or any range therein. In one embodiment, the localization of agent in an implant during such foam molding can be obtained by varying the amount and/or type of agent in the solid polymeric materials while adding the solid polymeric materials to the foam mold as desired such that the agent becomes localized in the formed implant.

In other embodiments, a stent is formed by injection molding or extrusion of a tube followed by cutting a pattern of a stent into the tube. In these embodiments, a mixture of polymer and agent can be added prior to injection molding or extrusion or, in the alternative, the agent can be absorbed by the stent after the stent has been formed.

Forming a Coating

In some embodiments of the invention, the compositions are in the form of coatings for medical devices such as, for example, a balloon-expandable stent or a self-expanding stent. There are many coating configurations within the scope of the present invention, and each configuration can include any number and combination of layers. In some embodiments, the coatings of the present invention can comprise one or a combination of the following four types of layers:

(a) an agent layer, which may comprise a polymer and an agent or, alternatively, a polymer free agent;

(b) an optional primer layer, which may improve adhesion of subsequent layers on the implantable substrate or on a previously formed layer;

(c) an optional topcoat layer, which may serve as a way of controlling the rate of release of an agent; and

(d) an optional biocompatible finishing layer, which may improve the biocompatibility of the coating.

In many embodiments, each layer can be applied to an implantable substrate by any method including, but not limited to, dipping, spraying, pouring, brushing, spin-coating, roller coating, meniscus coating, powder coating, inkjet-type application, controlled-volume application such as drop-on-demand, or a combination thereof. In these embodiments, a dry coating containing a biodegradable polymer may be formed on the stent when the solvent evaporates. In some embodiments, at least one of the layers can be formed on a stent by dissolving one or more biodegradable polymers, optionally with a non-biodegradable polymer, in one or more solvents, and either (i) spraying the solution on the stent or (ii) dipping the stent in the solution.

In other embodiments, a medical device, such as a stent, can be coated with a polymeric material using methods that may include sputtering and gas-phase polymerization. Sputtering is a method that includes placing a polymeric material target in an environment that is conducive to applying energy to the polymeric material and sputtering the polymeric material from the target to the device to form a coating of the polymeric material on the device. Similarly, a gas-phase polymerization method includes applying energy to a monomer in the gas phase within an environment that is conducive to formation of a polymer from the monomer in the gas phase, and wherein the polymer formed coats the device.

In some embodiments, the agent layer can be applied directly to at least a part of an implantable substrate as a pure agent to serve as a reservoir for at least one bioactive agent. In another embodiment, the agent can be combined with a polymer, biodegradable or durable, as a matrix, wherein the agent may or may not be bonded to the polymer. In another embodiment, an optional primer layer can be applied between the implantable substrate and the agent layer to improve adhesion of the agent layer to the implantable substrate and can optionally comprise an agent.

In other embodiments, a pure agent layer can be sandwiched between layers comprising biodegradable polymer. In other embodiments, the optional topcoat layer can be applied over at least a portion of the agent layer to serve as a topcoat to assist in the control the rate of release of agents and can optionally comprise an agent. In another embodiment, a biocompatible finishing layer can be applied to increase the biocompatibility of the coating by, for example, increasing acute hemocompatibility, and this layer can also comprise an agent.

Forming IC Profiles

The polymeric matrices taught herein can be a ternary system having an agent, polymer, and solvent; and, the relationship between the elements in this ternary system can affect the IC profiles obtained within the polymeric matrices. An example of the type of relationship that can affect the IC profile is the relative hydrophobicity and hydrophilicity of the three components in a given polymeric matrix. A fourth factor to consider in developing an IC profile can be variations in the boundary conditions that can be present during processing of a polymeric matrix used in a medical device or coating. Boundary conditions can be varied at each step in the process of forming a medical device or coating and include, but are not limited to, pressure, temperature, and atmosphere, wherein the atmosphere can include, but is not limited to, relative humidity, solvent vapor, or a combination thereof. Because of these boundary considerations, process applications such as the application of an external pressure, temperature, or a combination thereof such as, for example, freeze-drying can alter the IC profile and serve as a means to design a predetermined IC profile for a desired release rate of an agent.

The polymer matrix can include not only polymers but also polymers combined with ceramics and/or metals, which can also affect the relationship between the elements in the system. Examples of ceramics include, but are not limited to, hydroxyapatite, BIOGLASS®, and absorbable glass. Examples of metals include, but are not limited to magnesium, copper, titanium, and tantalum.

The compositions of the present invention can be used for one or any combination of layers, and a layer may comprise one or more IC profiles that may include, for example, selectively-placed agents within a desired IC profile at a predetermined region on a medical device or within a coating. In some embodiments, any of the polymers taught herein can be used as one of the layers or can be blended or crosslinked with the compositions in the embodiments taught herein.

In some embodiments, the methods of the present invention can be used to coat a medical device with layers formed from polymeric matrices having one or more IC profiles. In some examples, the IC profiles can include a pure agent as a layer within a combination of layers, such that the IC profile represents a maximum agent concentration.

In other embodiments, droplets can be formed from a combination of an agent and a polymer that is applied within a combination of layers, wherein each layer may otherwise have its own concentration of agent, and the combination of layers forms an IC profile. In these embodiments, droplets can be formed from agents encapsulated by a polymer, and the encapsulation can provide an additional control over the release of the agent, protect the agent to improve shelf-life, or a combination thereof. In other embodiments, the encapsulated agent can be pure, blended with a polymer, connected to a polymer, or a combination thereof.

In some embodiments, droplets such as those described above can be formed and applied as a suspension within a coating composition, and the coating composition can be applied using any coating method described above such as, for example, spraying, dipping, and controlled-volume formation, to name a few. In controlled volume formation, a droplet can be encapsulated within a larger droplet for a staged release of one or more agents. In these embodiments, the droplets can be formed in various sizes, wherein the sizes can vary due to the amount of agent, amount of encapsulating polymer, or a combination thereof.

In other embodiments, the droplets can be sandwiched between one or more layers that can be formed from droplets or from more traditional coating techniques such as, for example, spraying or dipping. It should be appreciated that these embodiments are not limited to coatings, since the droplets can be formed and dispersed in a polymeric composition that has been designed to form the structure of a medical device.

FIGS. 7a and 7b illustrate a sandwiched-coating design according to some embodiments of the present invention. FIG. 7a illustrates a cross-section of a stent strut 701 in which the abluminal surface 702 includes a first layer 703 containing agent B applied to the abluminal surface 702 and a second layer 704 containing agent A applied on the first layer 703 containing agent B. Each of the layers can be formed by any method known to one of skill in the art including, but not limited to, any one or any combination of the methods described above, and the layers can be applied to the entire stent or select regions of the stent.

In some embodiments, the first layer 703 can have an IC profile that is different from an IC profile in the second layer 704, such that agents A and B are delivered at different release rates, wherein the assumption can be that the difference between diffusion coefficients of the first layer 703 and second layer 704 is negligible. FIG. 7b illustrates a cross-section of the stent strut 701 in which the first layer 703 and the second layer 704 are coated by a third layer 705. The third layer 705 can contain any composition taught herein such as, for example, a topcoat to assist in controlling the rate of release of the agents, act as a biobeneficial layer, deliver one or more agents, or a combination thereof.

In some embodiments, each layer within the combination of layers can have a unique IC profile for each of the one or more agents, such that the combination of layers provides a controlled delivery of the one or more agents in a subject. In other embodiments, the combination of layers provides a step-by-step gradient of IC profiles, the sum of which provides an overall IC profile of one or more agents within a medical device, coating, or a combination thereof.

FIG. 8 illustrates a checkerboard-type coating design by showing a top view of an abluminal surface of a stent that was coated in sections according to some embodiments of the present invention. The process of coating the abluminal surface 801 of the stent in sections 802 can occur simultaneously or as a series of coating steps. Each section 802 of the checkerboard-type coating design can have a unique IC profile of one or more agents. In one example, each of the sections 802 can contain a single agent, more than one agent, or a combination thereof. In another example, each section 802 can contain an IC profile that is similar or equal to the other sections 802. In another example, each section 802 contains an IC profile that is tailored to deliver a particular agent from a select region of a medical device such as, for example, a stent. In another example, each section 802 contains an IC profile that is similar to adjacent sections 802, but the release rate of agents can vary due to a variation in diffusion coefficients, for example, as a result of adding a biodegradable polymer in the polymeric matrix. In another example, each section 802 has a similar or equal thickness. In another example, each section 802 can vary in thickness due to any one or any combination of the above factors. The IC profiles can be developed using any method taught herein.

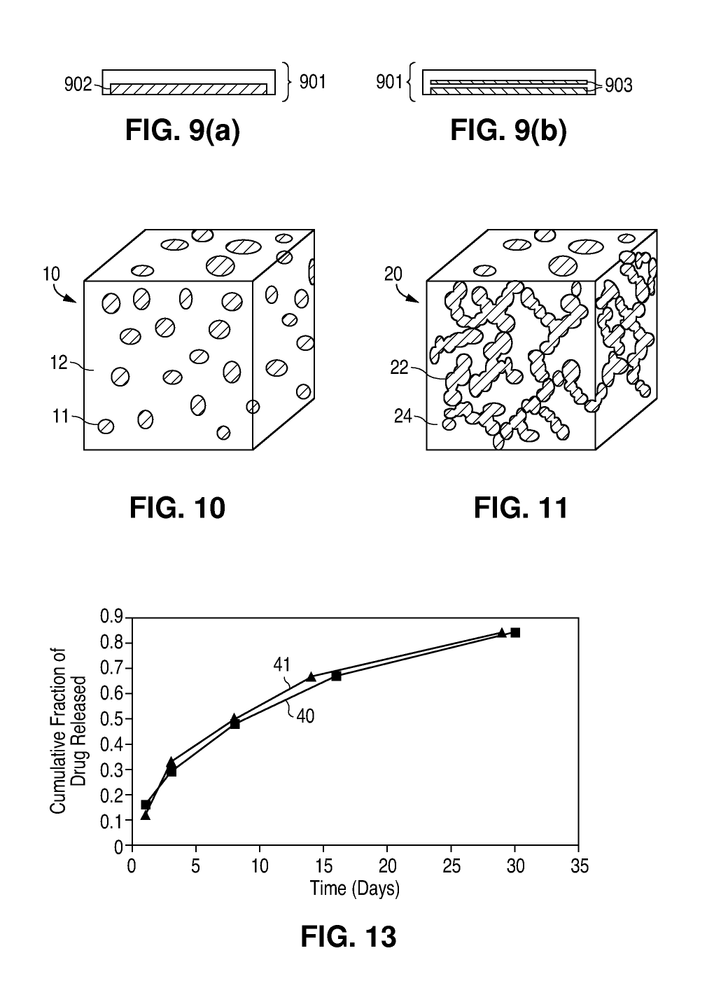

FIGS. 9a and 9b illustrate an engraved-type coating design by showing a top view of the abluminal surface of a stent with engravings according to some embodiments of the present invention. The engravings can be in any shape, size or form such as, for example, channels or pits. FIG. 9a shows a single channel 902 on the abluminal surface 901 of the stent, and FIG. 9b shows a parallel track-type coating design 903 on the abluminal surface 901 of the stent.

In some embodiments, a channel width can range from about 0.0005 inches to about 0.005 inches. In other embodiments, the channel width can range from about 0.001 inches to about 0.004 inches. In other embodiments, the channel width can range from about 0.001 inches to about 0.002 inches. In other embodiments, there can be a single pit. In other embodiments, the engravings can be continuous on the abluminal surface on each strut of the stent such as, for example, a continuous channel. In other embodiments, the engravings can be discontinuous and placed in select regions on the abluminal surface of the stent. In other embodiments, the stent can have a combination of any shape engravings such as, for example, a combination of channels and pits. The pits and channels can be formed using any method known to one of skill in the art such as, for example, laser cutting, extruding, or molding.

In many embodiments, the agents can be dissolved in the polymeric matrix exist in a dispersed phase within the polymeric matrix, or a combination thereof. In some embodiments, the agent component of a polymeric matrix can dissolve in a polymer phase, form a dispersed phase, or dissolve to its saturation point and concurrently form a dispersed phase, depending on factors including, but not limited to, the thermodynamic relationships between the agents and the polymers and the concentration of the agent in the polymeric matrix.

In many embodiments, the polymeric matrix can include a combination of polymers. Without intending to be bound by any theory or mechanism of action, an agent can be more thermodynamically stable in a first polymer than a second polymer, preferentially dissolve in the first polymer and create a first polymer/agent combination as a dispersed phase that can be substantially or completely immiscibility with the second polymer. In these embodiments, the second polymer can be referred to as a “bulk phase,” and the first polymer/agent combination can be referred to as an “agent-enriched phase.”

In some embodiments, an agent can have a preferential solubility in the solvent used to form the polymeric matrix, wherein the solvent preferentially solubilizes the second polymer over the first polymer. In these embodiments, an agent that ordinarily would preferentially dissolve in a first polymer can become preferentially incorporated in a second-polymer phase upon removing the solvent to form the medical device or coating.

An interconnected agent-enriched dispersed phase provides another means for affecting the diffusion coefficient and controlling the release of agents from a polymeric matrix. In many embodiments, an agent-enriched phase will reach a percolation threshold at a concentration of about 30% by volume within the combined volume of the polymer matrix and agent. The “percolation threshold” is the point at which the agent-enriched phase begins to connect with itself and form an interconnected network of the agent-enriched phase within the polymeric matrix. The percolation threshold is the point at which the agent-enriched phase forms its own channel for diffusion.

FIG. 10 illustrates a section of a polymeric matrix containing an agent-enriched phase at a concentration that is below about 30% by volume according to some embodiments of the present invention. The section 10 of the polymeric matrix is below the percolation threshold, since the agent-enriched phase 11 has not yet reached the concentration required to begin forming an interconnected network within the bulk phase 12 of the polymeric matrix.

FIG. 11 illustrates a section of a polymeric matrix containing an agent-enriched phase at a concentration that is above about 30% by volume according to some embodiments of the present invention. The section 20 of the polymeric matrix is above the percolation threshold, since the agent-enriched phase 22 has reached the concentration required to begin forming an interconnected network within the bulk phase 24 of the polymeric matrix.

In some embodiments, diffusion of an agent through an interconnected, agent-rich dispersed phase can result in a faster release of an agent. In other embodiments, the agent exists in both the interconnected, agent-enriched dispersed phase and the bulk phase, such that release of the agent occurs through diffusion across both phases.

The compositions described above can all include droplets of agents, agents blended and/or conjugated with a polymer, agents encapsulated with a polymer, or a combination thereof, according to some embodiments of the present invention. These droplets can be formed using any method known to one of skill in the art including, for example, methods that dispense droplets with a nozzle and methods that do not require a nozzle to dispense droplets. The methods that dispense droplets with a nozzle can include any source of pressure known to one of skill in the art.

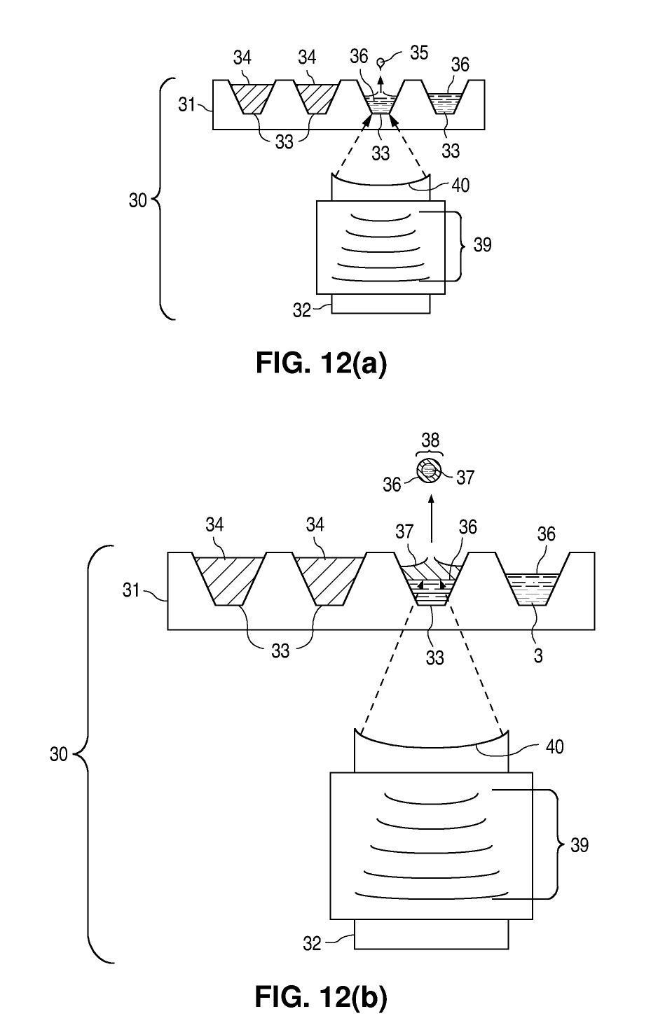

FIGS. 12a and 12b illustrate an ejector assembly that does not require a nozzle, according to some embodiments of the present invention. In some embodiments, the ejector assembly 30 can be used for controlled delivery of a coating composition that does not require a nozzle. FIG. 12a illustrates a cross section of the ejector assembly 30 comprising a reservoir housing 31 and a transducer 32. The transducer 32 outputs acoustic energy 39 at a reservoir 33 focused at the surface of the coating composition 34 therein. Each pulse ejects a known amount of the coating composition 34 in a droplet 35 from the reservoir 33 onto a medical device, thereby decreasing the coating composition 34 level in the reservoir 33. Accordingly, after each pulse of acoustic energy 39, the transducer 32 can be refocused to the new level in the reservoir 33 by a lens 40.

In an alternative embodiment, the reservoir 33 can be constantly refilled, thereby keeping the coating composition 34 level the same throughout the coating process. In some embodiments of the invention, the reservoirs 33 can each hold different coating substances. In one example, a first reservoir can hold a first coating composition 34 while a second reservoir can hold a second coating composition 36. The transducer 32 can then cause the ejection of different coating substances onto the medical device during a single coating process. Further, since there is no contact between the transducer 32 and reservoirs 33, the chance of cross contamination between reservoirs 33 is minimized or eliminated and there is no possibility of clogging any ejector assembly 30. It should be appreciated that nearly any number of compositions can be applied using this method.

In the embodiment shown in FIG. 12b, one or more of the reservoirs 33 may contain two different coating substances: a first substance 36 and a second substance 37, such that the transducer 33 can eject a combined drop 38 from the reservoir 33 by focusing a pulse of acoustic energy 39 at the interface between the two substances. The pulse of acoustic energy 39 is focused by the lens 40. Accordingly, in some embodiments, the medical device can be coated simultaneously with two different coating substance, such as a first substance 36 encapsulating a second substance 37. In some embodiments, the first substance 36 can be a biodegradable polymer selected to control the release of second substance 37, which can be a desired bioactive agent. In other embodiments, the first substance 36 can be a first agent, and the second substance 37 can be a second agent, wherein the agents can be any agent taught herein.

An advantage of the ejector assembly 30 illustrated in FIGS. 12a and 12b is the improved ability to eject controlled volumes, such as droplets, in a true “drop-on-demand,” or “monodispersed” form. In some embodiments, the controlled-volumes can be delivered drop-by-drop in specific locations. In other embodiments, the controlled volumes can be delivered in a continuous string using, for example, high frequency acoustic energy.

The controlled-volumes can be delivered in a variety of sizes. In some embodiments, the controlled-volumes can be dispersed in volumes that range from about 1 femtoliter to about 1 microliter, from about 1 femtoliter to about 100 nanoliters, from about 1 femtoliter to about 10 nanoliters, from about 10 femtoliters to about 0.1 nanoliters, from about 10 femtoliters to about 100 picoliters, from about 100 femtoliters to about 10 picoliters, and any range therein. In some embodiments, the controlled-volume is smaller than 10 picoliters to assist in even distribution of monodisperse droplets. An advantage of this broad range of controlled-volumes is that extremely potent agents can be delivered alone in the desired quantities to a desired area on a surface of a medical device. Another advantage of this broad range of controlled-volumes is that multiple agents can be delivered independently, or in combination, in a range of quantities to a range of desired areas and on multiple surfaces of a medical device.

It should be appreciated that a process of forming a medical article or coating can include additional process steps such as, for example, the use of energy such as heat, electromagnetic radiation, electron beam, ion or charged particle beam, neutral-atom beam, and chemical energy. The process of drying can be accelerated by using higher temperatures. In some embodiments, the control of the application of energy includes manual control by the operator. In other embodiments, the control of the application of energy includes a programmable heating control system. In some embodiments, the application of energy can result in a coating composition temperature that ranges from about 35° C. to about 100° C., from about 35° C. to about 80° C., from about 35° C. to about 55° C., or any range therein. In some embodiments, any procedure for drying or curing known to one of skill in the art is within the scope of this invention.

A medical article or coating can also be annealed to enhance the mechanical properties of the composition. Annealing can be used to help reduce part stress and can provide an extra measure of safety in applications such as complex medical devices, where stress-cracking failures can be critical. The annealing can occur at a temperature that ranges from about 30° C. to about 200° C., from about 35° C. to about 190° C., from about 40° C. to about 180° C., from about 45° C. to about 175° C., or any range therein. The annealing time can range from about 1 second to about 60 seconds, from about 1 minute to about 60 minutes, from about 2 minute to about 45 minutes, from about 3 minute to about 30 minutes, from about 5 minute to about 20 minutes, or any range therein. The annealing can also occur by cycling heating with cooling, wherein the total time taken for heating and cooling is the annealing cycle time.

The following examples are provided to further illustrate embodiments of the present invention.

### EXAMPLE 1

A lumped-parameter mass transport model was developed to predict the rate of release of agents from a coating. As described above, it was assumed that the dissolution and diffusion of an agent within a polymeric matrix can be lumped into an effective diffusivity and describes the mass transport of the agent within the coating. It was also assumed that the transport of the agent in the coating may occur through Fickian diffusion, as derived and described above. Using these assumptions, the transport of the agent through a polymeric matrix can be predicted by, for example, the following system of equations:

$$\frac{\partial \overline{C}}{\partial \overline{t}} = \frac{{\partial }^{2} \overline{C}}{\partial {\overline{x}}^{2}}$$

$$IC \text{:} \overline{C} = f ( \overline{x} ) for 0 \leq \overline{x} \leq 1$$

$$BC 1 \text{:} {\frac{\partial \overline{C}}{\partial \overline{x}} \vert}_{\overline{t} , 0} = 0$$

$$BC 2 \text{:} {\frac{\partial \overline{C}}{\partial \overline{x}} \vert}_{\overline{t} , 1} = - \frac{{K}_{m} L}{D} ( {K \overline{C} \vert}_{\overline{t} , 1} - {\overline{C} \vert}_{\overline{t} , bulk} ) ;$$

where, in this example,

t is time in sec;

t̅ is dimensionless time ( t̅=t/(L²/D));

L is a thickness of the coating in cms;

D is a diffusivity in cm²/sec;

x̅ is a dimensionless length (actual length/L);

C̅ is a dimensionless concentration;

C̅| _{t̅,1} is a dimensionless concentration at the surface of the coating at any time;

C̅| _{t̅,bulk} is a dimensionless concentration outside the stent coating at any time;

Kₘ is a mass transfer coefficient in (cm/sec); and,

K is a dimensionless partition coefficient at equilibrium.

Generally speaking, the mass of the agent in the polymeric matrix at any time t̅ is given by

$$M ( \overline{t} ) = {\int }_{0}^{1} C ( \overline{x} , \overline{t} ) A d \overline{x} = A {\int }_{0}^{1} C ( \overline{x} , \overline{t} ) d \overline{x} ;$$

where M is the amount of agent (in μg) in the polymeric matrix at any time t̅; and,

A is the stent surface area in (cm²).

For a matrix configuration containing a agent reservoir and a top coat, the amount of agent in the matrix at any time t is given by the following analytical model:

$$\frac{M}{{M}_{0}} = {\sum }_{n = 0}^{∞} {( - 1 )}^{n} \frac{4}{( 2 n + 1 ) π {x}^{*}} sin ( \frac{( 2 n + 1 ) π {x}^{*}}{2} ) ( 1 - exp ( - \frac{{( 2 n + 1 )}^{2} {π}^{2} Dt}{4 {L}^{2}} ) ) ;$$

$$where {x}^{*} = \frac{Agent reservoir thickness}{Total coating thickness ( L )} ;$$

Total coating thickness=(agent reservoir thickness)+(top-coat thickness); and,

M₀ is the initial amount of agent in the matrix.

FIG. 13 demonstrates the accuracy of fit for an analytical model used to predict release rates of agents from polymeric matrices according to some embodiments of the present invention. The cumulative amount of agent released according to model predictions was fit to published experimental data by iterating values of L²/D until a very good fit was obtained between the model prediction 40 and the in vivo experimental data 41; an example of a goodness-of-fit test known to one of skill in the art for such analyses is the Chi-Square Goodness-of-Fit test. The diffusivity was then calculated from this value of L²/D, since the coating thickness was known. The diffusivity was then used to compute the cumulative amount of agent released in-vivo for a clinically tested system. The in-vivo experimental data 41 fit well to the model predictions 40 using statistical methods known to one of skill in the art.

### EXAMPLE 2

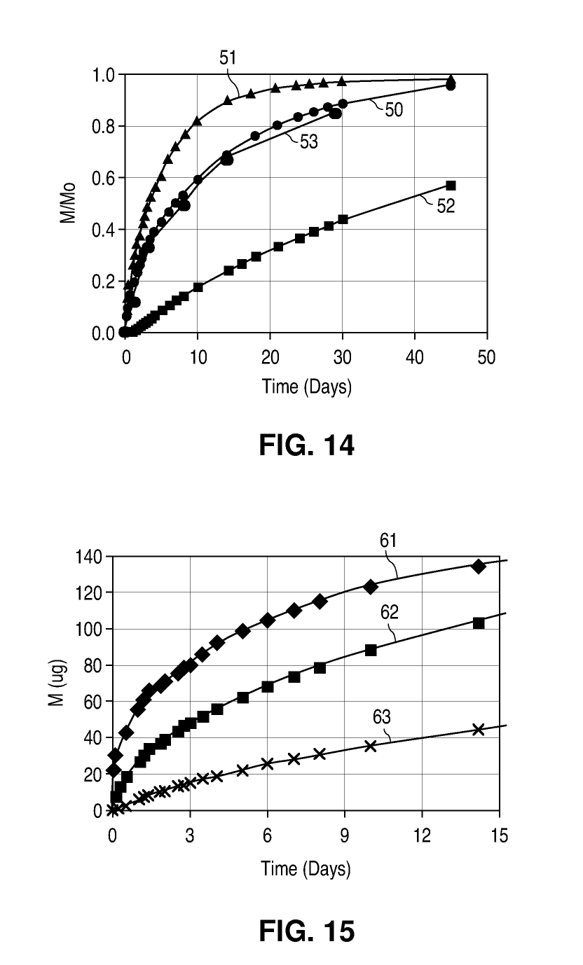

The agent diffusivity in the polymeric matrix provided valuable information for evaluating and predicting the effects of coating design parameters on agent release. FIG. 14 shows the fraction of agent released as a function of time for three different coating configurations according to some embodiments of the present invention. The different coating configurations were (1) a polymeric matrix reservoir (coating containing an agent) with no topcoat 51; (2) the same reservoir with a topcoat 52; and (3) the coating that provided the published experimental data 53 used to fit the model 50. The fastest release rate was observed for the polymeric matrix reservoir with no top coat 51. The addition of the topcoat lowers the release rate by acting as a rate limiting membrane.

The amount of agent released from a polymeric matrix is designated in FIG. 14 by “M”, and can be measured in vitro in a release medium. In the present example, the release medium was a buffered solution containing TRITON as a surfactant. The value of M as measured in the release medium can be verified by extracting the residual agent, “Ms” out of the spent or partially spent polymeric matrix, where M+Ms=Mo, and Mo is the initial amount of agent in the polymeric matrix.

Note that some losses in agent occur due to handling, degradation, etc., such that usually M+Ms<Mo. These losses should be taken into account in all calculations through standardization techniques, such as those known to one of skill in the art. One method of obtaining Mo is to extract all of the agent out of a polymeric matrix before any exposure of the matrix to a release medium and assign this value of Mo as the standardized value for that particular batch of polymeric matrices. The value of M for an in vivo system can be determined by measuring Mo and Ms, where M is the difference between those measured values.

The method was successfully applied to a stent coating (“reservoir”) containing poly(vinylidene fluoride-co-hexafluoropropylene) and everolimus at a dose of 100 μg/cm² to measure the release rates of the everolimus in vivo. The theoretical release rate results provided an excellent fit to the experimental release rate results over a 30 day release period. The fitting parameters from the 100 μg/cm² dose were used to evaluate the same stent coating having an additional heparin coating applied on top of the reservoir, as well as to subsequently predict several other doses. For example, the everolimus was loaded into the reservoir layer at 10 μg/cm², 20 μg/cm², and 45 μg/cm², and again an excellent fit between the theoretical release rate and the experimental release rate results were shown over a 30 day release period.

### EXAMPLE 3

Release rates for various IC profiles can be determined from the model calculation, which provides one of skill in the art with a means to design IC profiles within polymeric matrices of choice. The IC profiles described above represent the relationship between concentration and position within a polymeric matrix. In effect, each IC profile is a continuum of changing agent-to-polymer ratios, so an evaluation of the effect of agent-to-polymer ratios can be used to support the premise that control over the shape of an IC profile of an agent within a polymeric matrix can provide control over the release rates of the agent from the polymeric matrix.

FIG. 15 shows the effect of agent-to-polymer ratios on agent release from a polymeric matrix according to some embodiments of the present invention. A model system with a higher agent-to-polymer ratio 61 has a higher release rate than a model system with a lower agent-to-polymer ratio 62. A model system with lower agent-to-polymer ratio having a topcoat 63 further lowers the release rate.

This concept was applied to an in vivo test system using a polymeric matrix comprising poly(vinylidene fluoride-co-hexafluoropropylene) and everolimus as a homogeneous mixture coated on a stent to a thickness of about 5-6 μm. Theoretical and in vivo test results for a loading of about 100 μg/cm² and for a loading of 45 μg/cm² were compared. Not only did the in vivo results show an excellent correlation to the theoretical results in each case, but the difference in release rates were significant between the different loadings, where the 100 μg/cm² loading had a higher release rate than the 45 μg/cm² loading.

The discovery that control over agent-to-polymer ratios can provide control over agent release rates provides a basis for the development of coatings with one or more predetermined IC profiles. Factors affecting the IC profiles within a coating are discussed above and are further discussed below in the context of the ensuing examples.

Selection of Materials

The coating design process involves a careful selection of materials, which include, but are not limited to, one or more polymers and one or more agents. The combination of the one or more polymers and one or more agents often involves use of a solvent.

The design of an IC profile relies, at least partially, on the behavior among the materials chosen and the resulting morphology of the polymeric matrix formed from those materials. The solubility parameters of the one or more polymers that are chosen, for example, can provide an indication of the solubility of the polymers in a solvent of choice and the miscibility between the polymers. Likewise, the solubility of the one or more agents in a particular solvent or solvent/polymer system can also be a consideration, as well as the miscibility between the one or more agents and the one or more polymers. Such considerations can help one of skill in the art to design a system while having control over the phase morphology of the system.

In one example, a system may be chosen to include polymers in dispersed phases at a percolation threshold to provide the desired agents with channels for release from the system. In this case, polymers with solubility parameters that differ enough to form separate phases would be chosen and would be combined in an appropriate ratio to reach the percolation threshold.

In another example, a system may be chosen to include an agent that is much more miscible in a particular polymer within a combination of polymers, such that the agent is primarily present in that polymer in the system. In another example, a system may be chosen such that a polymer in a combination of polymers has a much higher solubility in a particular solvent, such that the solvent can carry the agent into the polymer as the solvent is removed from the system. In another example, a system may be chosen such that the agent can be dissolved and dispersed relatively evenly throughout a polymeric matrix containing a combination of polymers regardless of whether there is a dispersed phase within the combination of polymers. In another example, other process considerations such as time, temperature, and pressure, and their effects on the behavior among the select materials are integral to the selection of the materials to use in a system. In another example, a combination of the concepts taught in this example can be used to create a coating design with a combination of morphology characteristics.

### EXAMPLE 4

A polymeric matrix of everolimus can be dispersed in poly(D,L-lactide) to serve as an example, wherein the application of a thin topcoat of poly(D,L-lactide) on the polymeric matrix containing everolimus will effectively slow down the release rate of the everolimus from the polymeric matrix significantly as a result of the hydrophobic nature of the poly(D,L-lactide).

A hydrophobic agent such as, for example, paclitaxel can be encapsulated in a hydrophobic polymer or copolymer such as, for example, a poly(styrene-co-isobutylene-co-styrene) triblock copolymer. At a given loading of agent, the release rate of agent from such a combination can be significantly lower than that of an agent from a more hydrophilic polymer such as, for example, poly(ethylene-co-vinyl alcohol).

Selection of Process Conditions

As discussed above, the IC profile can be depend on a variety of process conditions, which include, for example, the way a composition is applied, dried, and possibly annealed. Forming a medical device or coating with a desired IC profile can include creating the IC profile as the compositions containing agent are applied. In one example, an IC profile of a coating can be developed one pass at a time. The agent concentration can be increased or decreased on each pass to create any IC profile that may be desired, wherein the IC profile can be a relatively continuous distribution of agent. Such a distribution may provide a release profile with smooth, or substantially smooth, transitions in agent release rates.

The desired IC profile can be any one or any combination of profiles. It should be appreciated that other process conditions such as, for example, the time, temperature, and pressure of subsequent steps such as, for example, drying can alter the IC profile. A freeze drying or critical-point drying process may be chosen, for example, to remove solvent without altering the IC profile. In another example, a series of layers can be applied to develop an IC profile, wherein each layer can be applied through multiple passes and have a constant IC profile that differs from adjacent layers. The resulting medical device or coating would contain a series of layers that provide an incremental distribution of agent. Such a distribution may provide a release profile that has distinct changes, such as steps, in agent release rates. These steps can be large, small, or a combination thereof, by design.

Forming a medical device or coating with a desired IC profile can include creating the IC profile after the compositions containing agent have been applied. In one example, an agent that migrates with a solvent can be profiled by controlling the rate of solvent migration. The rate of solvent migration can be controlled by altering the pressure and/or temperature in the environment of a solvent removal process such as, for example, drying. Such control of the pressure and/or temperature can allow for indirect control of the pattern that is taken by an agent concentration relative to position in a polymeric matrix. The IC profiles can then be designed to take on virtually any profile desired such as, for example, a predetermined wave profile that can provide a pulsed administration of a desired agent.

### EXAMPLE 5

The development of IC profiles can implement boundary condition control through, for example, use of solvent vapor, humidity, temperature, and/or pressure to establish a diffusion medium for an agent in a polymeric matrix. The establishment of a diffusion medium allows for the mobility of agent during processing of the polymeric matrix.

A stent can be coated with a hydrophobic agent layer that is subsequently coated with a hydrophilic polymeric matrix. Movement of the underlying hydrophobic agent layer through the hydrophilic polymeric matrix would not normally be thermodynamically favorable. However, the agent can be drawn through the hydrophilic polymeric matrix through the use of a boundary condition containing solvent vapor that can permeate the hydrophilic polymeric matrix and serve as a diffusion medium for the hydrophobic agent. The movement of the agent can also be influenced by administration of pressure and/or heat, and this administration can be constant, cyclic, or any variation discovered by one of skill in the art to create an IC profile that will provide a desired release rate of the agent in vivo. The distribution of the agent can also have a chromatographic effect that can be altered through the selection of polymers, copolymers, metals, ceramics, additional agents combined with the foregoing, and the like. Likewise, it should be appreciated that the inverse of this example can be used to move any agent through any polymeric matrix such as, for example, the use of a high relative humidity to move a hydrophilic agent through a hydrophobic polymeric matrix.

### EXAMPLE 6

A DES system can be coated in a series of layers using a very low agent-to-polymer ratio for each layer. A very low agent-to-polymer ration can range, for example, from about 1/10 to about 1/50.

A theoretical modeling of the general profile illustrated in FIG. 3b was compared to a theoretical modeling of the inverse of that IC profile, where the assumption was that the same composition and process conditions would be employed and the diffusion coefficient would be the same or substantially the same. No topcoat was applied to either profile in this theoretical modeling study. FIG. 3b illustrates a positive slope, which indicates that the region of highest agent concentration is at the surface of the coating, and the region of lowest concentration would be at the surface of the medical device. The inverse of that profile would be a negative slope, which would indicate that the region of highest concentration would be at the surface of the medical device, and the region of lowest concentration would be at the surface of the coating. The theoretical results showed a dramatic difference in release rates, where the positive slope illustrated in FIG. 3b had a much higher release rate than the negative slope.

The IC profiles can be obtained by varying the agent concentration in each pass, or by varying the agent concentration in each layer, which can be a series of passes. A 12 mm stent can be coated using a first pass with an agent-to-polymer ratio of about 1/10 for application of the the first 200 μg, about 1/30 for application of the next 200 μg, and finally about 1/50 for application of an additional 200 μg. The effective diffusivity will remain constant in this example because of the low overall agent-to-polymer ratio. The progressive reduction in the ratio will result in an IC profile that has an initial release rate that is slow but sustainable when compared to a corresponding flat IC profile for the exact same dose.

Measuring IC Profiles

The IC profiles can be measured using confocal techniques that have been used in tissue culture and vascular grafts for nondestructive imaging of 3D distributions. The methods include, but are not limited to, optical sectioning of a layer as a function of depth to acquire a signal that can be reconstructed for a 3D image. The techniques that can be used include, but are not limited to, laser confocal raman microscopy, confocal fluorescence microscopy, and imaging fourier-transform infrared microscopy.

### EXAMPLE 7

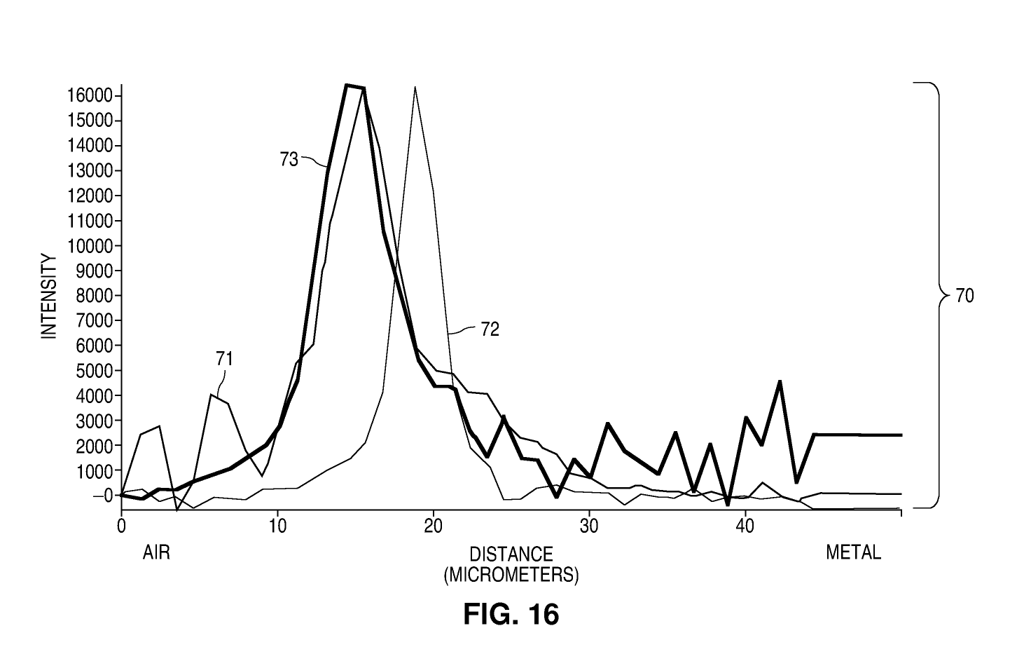

FIG. 16 illustrates a graphical representation of a coating profile measurement that correlates point component concentration with depth according to some embodiments of the present invention. A coating design containing poly(D,L-lactide), everolimus, and parylene was applied to a metal stent. The profile of each component within the coating was then determined using Laser Confocal Raman Microscopy.

Three spectra are provided in FIG. 16 and are overlayed to represent the concentration profile 70 across the thickness of the coating on the stent. The intensity of a data point can be directly related to a concentration at that depth across the thickness of the film, where a depth of zero is the air/coating interface. The outer coating spectra 71 contains the poly(D,L-lactide), the inner coating spectra 72 contains the parylene, and agent spectra 73 contains the everolimus.

### EXAMPLE 8

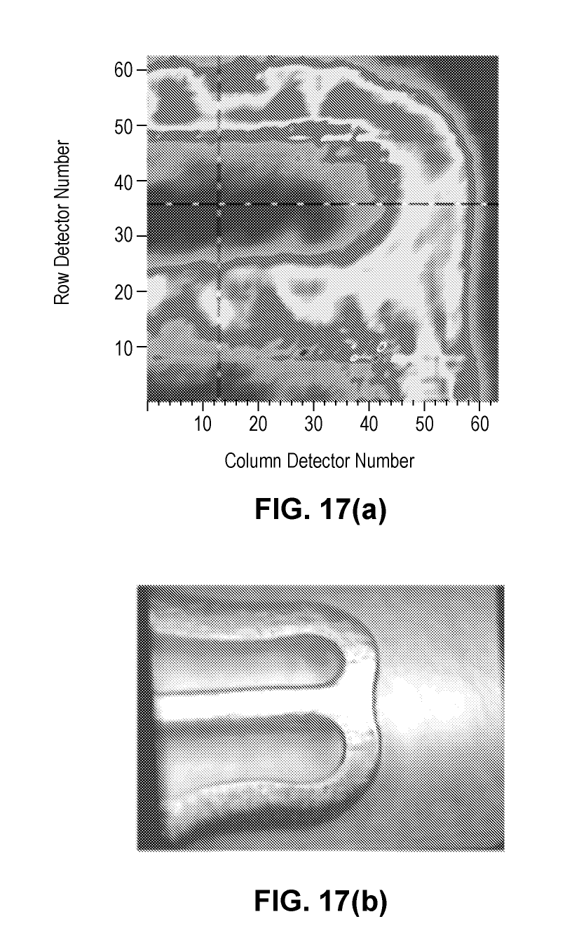

FIGS. 17a and 17b illustrate a graphical representation of a coating profile measurement that correlates bulk component concentration with position on the stent according to some embodiments of the present invention. In this example, a coating design on a CHAMPION drug-eluting stent was measured for a bulk agent concentration profile of everolimus throughout the polymeric matrix using a Digilab STINGRAY focal planar imaging FTIR. The concentration gradient profile across the bulk of a coating was shown using a color gradient in this example from red . . . to yellow . . . to green . . . to blue, where red is the highest concentration of agent, and blue is the area of zero concentration outside the stent.

In FIG. 17a the bulk concentration profile can be determined by looking at the color-scale of the image produced by the imaging FTIR technique. FIG. 17b illustrates the area of a stent strut that is being profiled. Areas in FIG. 17a that contain a high everolimus concentration are red, whereas areas of low everolimus concentration are blue.

Since energy absorption is proportional to agent density using the focal plane imaging FTIR, a measure of absorbance units (AU) represents agent density in a particular location on a stent. The following table of measurement data summarizes the average agent absorbance peak height (i.e. agent density) in AU as a function of (1) position on a stent and (2) a bulk absorbance unit for the whole stent. These color densities can be calibrated to numerical concentration profiles, such as those provided in the table for 7 different locations on three different CHAMPION stents. The concentration profiles in the table can be used to determine the deviation in agent concentration in select regions of a stent by providing an average and standard deviation for the select regions.

CHAMPION STENT BULK CONCENTRATION PROFILING

| | Concentration | | | | | | | |
|---|---|---|---|---|---|---|---|---|
| | (absorbance | | | | | | | |
| | units, AU) | | | | | | | |
| | and | | | | | | | |
| | Relative | | | | | | | |
| | Standard | | | | | | | Middle- |
| | Deviation | Distal | Distal- | | Middle- | Proximal | Middle- | Counter |
| Stent | (% RSD) | End | Middle | Middle | Proximal | End | Clockwise | Clockwise |
| A | AU | 0.22 | 0.83 | 0.53 | 0.47 | 0.47 | 0.64 | 0.42 |
| | % RSD | 27% | 23% | 29% | 29% | 34% | 20% | 11% |
| B | AU | 0.29 | 0.59 | 0.40 | 0.43 | 0.16 | 0.45 | 0.49 |
| | % RSD | 24% | 19% | 25% | 15% | 58% | 38% | 17% |
| C | AU | 0.11 | 0.53 | 0.58 | 0.64 | 0.52 | 0.37 | 0.49 |
| | % RSD | 17% | 31% | 30% | 17% | 30% | 18% | 44% |
| Average | AU | 0.21 | 0.65 | 0.50 | 0.52 | 0.38 | 0.48 | 0.47 |
| | % RSD | 23% | 25% | 28% | 21% | 43% | 27% | 28% |

| | |
|---|---|
| Bulk Average ± | 0.46 ± 29% |
| % RSD | |

Bulk concentration profiles can be designed and confirmed in order to design the delivery of high and low release rates from select areas of a medical device using the methods of the present invention. These profiles can provide for control over many factors in the design of medical devices and coatings, and these factors include, but are not limited to, the rate release of agents as well as other physical parameters such as water uptake, percolation, relative diffusivities, and the like, which can also be related to other physical performance parameters such as, for example, mechanical properties that include the toughness of a polymeric matrix.

### EXAMPLE 9

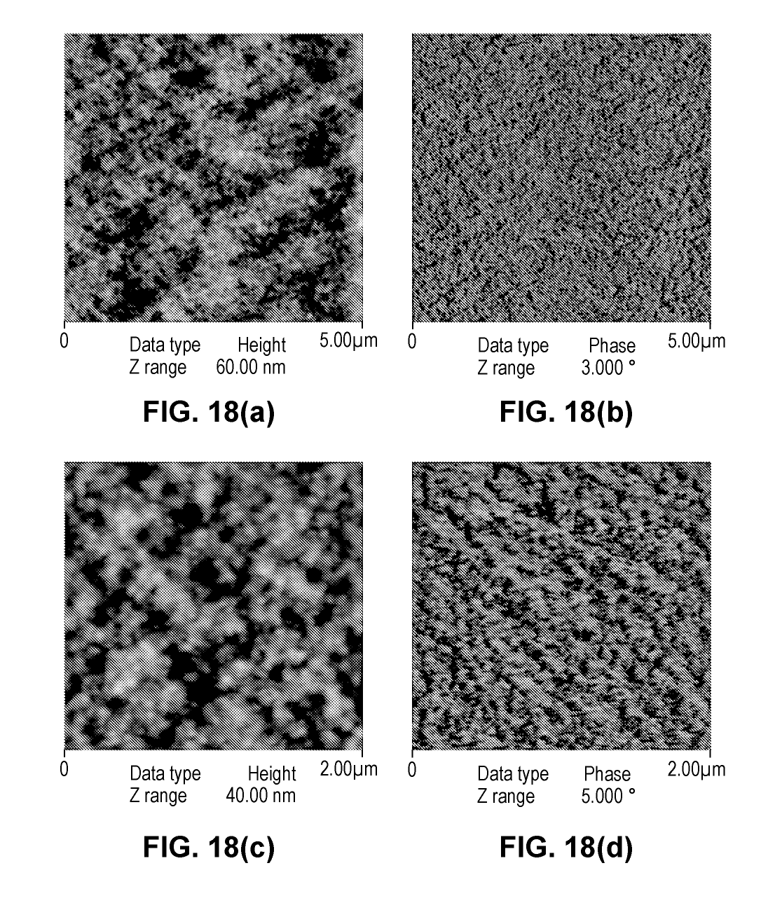

An understanding of the phase morphologies that are present in a particular polymeric matrix design can be important to forming a medical device or coating with an IC profile that produces a desired release rate. FIGS. 18(a)-(d) illustrates a pictorial representation of a coating profile measurement that correlates component distribution with depth according to some embodiments of the present invention.

In this example, a coating design containing poly(vinylidene fluoride-co-hexafluoropropylene) (PVDF-HFP), everolimus, and poly(butyl methacrylate) (PBMA) was applied to a metal stent. The microstructure profile within the coating was then determined using Atomic Force Microscopy. FIG. 18 shows a homogeneous phase distribution, which indicates that the agent was totally soluble in the polymer matrix and has no phase separation.

Microtoming cross sectional surface topography is shown in FIGS. 18(a) and 18(c), whereas phase imaging of a DES coating using AFM is shown in FIGS. 18(b) and 18(d) and illustrate homogeneous distribution of an agent in polymer. In homogenous polymeric matrix designs, the agents can be released by diffusion control through channels such as, for example, fluid filled pores that may be formed when fluid flows in from the surface of the device to replace agents that have been released.

While particular embodiments of the present invention have been shown and described, those skilled in the art will note that variations and modifications can be made to the present invention without departing from the spirit and scope of the teachings. A multitude of embodiments that include a variety of chemical compositions, polymers, agents and methods have been taught herein. One of skill in the art is to appreciate that such teachings are provided by way of example only and are not intended to limit the scope of the invention.

We claim:

1. A method of coating a medical device, the method comprising:
providing a polymer and an agent;
providing a solvent;
providing the medical device, the medical device having a surface;
forming a solution or dispersion of the polymer and the agent and optionally other materials;
applying the solution or dispersion to the surface of the medical device;
removing the solvent by freeze drying or critical point drying; and
repeating the operations of applying the solution or dispersion to the surface of the medical device and removing the solvent by freeze drying or critical point drying until a desired coating thickness is achieved.

2. The method of claim 1 wherein the polymer is selected from a group consisting of polyesters, poly(hydroxyalkanoates) (PHAs), poly(ester amides), poly(ethylene glycol) (PEG), polycaprolactones, poly(D-lactide), poly(L-lactide), poly(D,L-lactide), poly(D,L-lactide-co-PEG), poly(D,L-lactide-co-trimethylene carbonate), polyglycolides, poly(lactide-co-glycolide), polydioxanones, polyorthoesters, polyanhydrides, poly(glycolic acid-co-trimethylene carbonate), polyphosphoesters, polyphosphoester urethanes, poly(amino acids), polycyanoacrylates, poly(trimethylene carbonate), poly(imino carbonates), polycarbonates, polyurethanes, copoly(ether-esters), polyalkylene oxalates, polyphosphazenes, PHA-PEG, poly(tyrosine carbonates), poly(tyrosine arylates), polyanhydrides, poly(hydroxyethyl methacylate), poly(N-acylhydroxyproline)esters, poly(N-palmitoyl hydroxyproline)esters, polyphosphazenes, poly(vinylidene fluoride-co-hexafluoropropylene), and any prodrugs, codrugs, metabolites, analogs, homologues, congeners, derivatives, salts, and combinations thereof.

3. The method of claim 1, wherein the agent comprises a component selected from a group consisting of bioactive agents, biobeneficial agents, diagnostic agents, plasticizing agents, and any prodrugs, codrugs, metabolites, analogs, homologues, congeners, derivatives, salts, and combinations thereof.

4. The method of claim 1, wherein the agent comprises a component selected from a group consisting of poly(alkylene glycols), phosphorylcholine, poly(N-vinyl pyrrolidone), poly(ethylene oxide), poly(acrylamide methyl propane sulfonic acid), poly(styrene sulfonate), polysaccharides, poly(ester amides), peptides, non-thrombotics, antimicrobials, nitric oxide donors, free radical scavengers, and any prodrugs, codrugs, metabolites, analogs, homologues, congeners, derivatives, salts, and combinations thereof.

5. The method of claim 4, wherein the poly(alkylene glycol) comprises a component selected from a group consisting of poly(ethylene glycol), polypropylene glycol), and any prodrugs, codrugs, metabolites, analogs, homologues, congeners, derivatives, salts, and combinations thereof.

6. The method of claim 4, wherein the polysaccharide comprises a component selected from a group consisting of carboxymethylcellulose, sulfonated dextran, sulfated dextran, dermatan sulfate, chondroitin sulfate, hyaluronic acid, heparin, hirudin, and any prodrugs, codrugs, metabolites, analogs, homologues, congeners, derivatives, salts, and combinations thereof.

7. The method of claim 4, wherein the peptide comprises a component selected from a group consisting of elastin, silk-elastin, collagen, atrial natriuretic peptide (ANP), Arg-Gly-Asp (RGD), and any prodrugs, codrugs, metabolites, analogs, homologues, congeners, derivatives, salts, and combinations thereof.

8. The method of claim 4, wherein the free radical scavenger comprises a component selected from a group consisting of 2,2′,6,6′-tetramethyl-1-piperinyloxy, free radical; 4-amino-2,2′,6,6′-tetramethyl-1-piperinyloxy, free radical; 4-hydroxy-2,2′,6,6′-tetramethyl-piperidene-1-oxy, free radical; 2,2′,3,4,5,5′-hexamethyl-3-imidazolinium-1-yloxy methyl sulfate, free radical; 16-doxyl-stearic acid, free radical; superoxide dismutase mimic; and any prodrugs, codrugs, metabolites, analogs, homologues, congeners, derivatives, salts, and combinations thereof.

9. The method of claim 4, wherein the nitric oxide donor comprises a component selected from the group consisting of S-nitrosothiols, nitrites, N-oxo-N-nitrosamines, substrates of nitric oxide synthase, diazenium diolates, and any prodrugs, codrugs, metabolites, analogs, homologues, congeners, derivatives, salts, and combinations thereof.

10. The method of claim 1, wherein the agent comprises a component selected from a group consisting of rapamycin, methyl rapamycin, everolimus, pimecrolimus, 42-Epi-(tetrazoylyl)rapamycin (zotarolimus, ABT-578), tacrolimus, and any prodrugs, codrugs, metabolites, analogs, homologues, congeners, derivatives, salts, and combinations thereof.

11. The method of claim 1, wherein the agent comprises a component selected from a group consisting of imatinib mesylate, paclitaxel, docetaxel, midostaurin, and any prodrugs, codrugs, metabolites, analogs, homologues, congeners, derivatives, salts, and combinations thereof.

12. The method of claim 1, wherein the agent comprises a component selected from a group consisting of estradiol, clobetasol, idoxifen, tazarotene, and any prodrugs, codrugs, metabolites, analogs, homologues, congeners, derivatives, salts, and combinations thereof.

13. The method of claim 1, wherein the agent comprises a combination of agents selected from a group consisting of everolimus and clobetasol; tacrolimus and rapamycin; tacrolimus and everolimus; rapamycin and paclitaxel; and combinations thereof.

14. The method of claim 1, wherein the medical device is an implantable medical device.

15. The method of claim 14, wherein the implantable medical device is a stent.

16. The method of claim 1, wherein the polymer is a biodegradable polymer.
